SEQUENCE LISTING (21 sequences)

SEQ ID NO: 1 - protein, 382 aa, Escherichia coli
MYALTQGRIFTGHEFLDDHAVVIADGLIKSVCPVAELPPEIEQRSLNGAILSPGFIDVQLNGCGGVQFNDTAEAVSVETLEIMQKANEKSGCTNYLPTLITTSDELMKQGVRVMREYLAKHPNQALGLHLEGPWLNLVKKGTHNPNFVRKPDAALVDFLCENADVITKVTLAPEMVPAEVISKLANAGIVVSAGHSNATLKEAKAGFRAGITFATHLYNAMPYITGREPGLAGAILDEADIYCGIIADGLHVDYANIRNAKRLKGDKLCLVTDATAPAGANIEQFIFAGKTIYYRNGLCVDENGTLSGSSLTMIEGVRNLVEHCGIALDEVLRMATLYPARAIGVEKRLGTLAAGKVANLTAFTPDFKITKTIVNGNEVVTQ

SEQ ID NO: 2 - protein, 367 aa, Escherichia coli
MKVKVLSLLVPALLVAGAANAAEVYNKDGNKLDLYGKVDGLHYFSDNKDVDGDQTYMRLGFKGETQVTDQLTGYGQWEYQIQGNSAENENNSWTRVAFAGLKFQDVGSFDYGRNYGVVYDVTSWTDVLPEFGGDTYGSDNFMQQRGNGFATYRNTDFFGLVDGLNFAVQYQGKNGNPSGEGFTSGVTNNGRDALRQNGDGVGGSITYDYEGFGIGGAISSSKRTDAQNTAAYIGNGDRAETYTGGLKYDANNIYLAAQYTQTYNATRVGSLGWANKAQNFEAVAQYQFDFGLRPSLAYLQSKGKNLGRGYDDEDILKYVDVGATYYFNKNMSTYVDYKINLLDDNQFTRDAGINTDNIVALGLVYQF

SEQ ID NO: 3 - protein, 516 aa, Escherichia coli
MQRRDFLKYSVALGVASALPLWSRAVFAAERPTLPIPDLLTTDARNRIQLTIGAGQSTFGGKTATTWGYNGNLLGPAVKLQRGKAVTVDIYNQLTEETTLHWHGLEVPGEVDGGPQGIIPPGGKRSVTLNVDQPAATCWFHPHQHGKTGRQVAMGLAGLVVIEDDEILKLMLPKQWGIDDVPVIVQDKKFSADGQIDYQLDVMTAAVGWFGDTLLTNGAIYPQHAAPRGWLRLRLLNGCNARSLNFATSDNRPLYVIASDGGLLPEPVKVSELPVLMGERFEVLVEVNDNKPFDLVTLPVSQMGMAIAPFDKPHPVMRIQPIAISASGALPDTLSSLPALPSLEGLTVRKLQLSMDPMLDMMGMQMLMEKYGDQAMAGMDHSQMMGHMGHGNMNHMNHGGKFDFHHANKINGQAFDMNKPMFAAAKGQYERWVISGVGDMMLHPFHIHGTQFRILSENGKPPAAHRAGWKDTVKVEGNVSEVLVKFNHDAPKEHAYMAHCHLLEHEDTGMMLGFTV

SEQ ID NO: 4 - protein, 351 aa, Escherichia coli
MKKSTLALVVMGIVASASVQAAEIYNKDGNKLDVYGKVKAMHYMSDNASKDGDQSYIRFGFKGETQINDQLTGYGRWEAEFAGNKAESDTAQQKTRLAFAGLKYKDLGSFDYGRNLGALYDVEAWTDMFPEFGGDSSAQTDNFMTKRASGLATYRNTDFFGVIDGLNLTLQYQGKNENRDVKKQNGDGFGTSLTYDFGGSDFAISGAYTNSDRTNEQNLQSRGTGKRAEAWATGLKYDANNIYLATFYSETRKMTPITGGFANKTQNFEAVAQYQFDFGLRPSLGYVLSKGKDIEGIGDEDLVNYIDVGATYYFNKNMSAFVDYKINQLDSDNKLNINNDDIVAVGMTYQF

SEQ ID NO: 5 - protein, 622 aa, Escherichia coli
MSTDNKQSLPAITLAAIGVVYGDIGTSPLYTLRECLSGQFGFGVERDAVFGFLSLIFWLLIFVVSIKYLTFVMRADNAGEGGILTLMSLAGRNTSARTTSMLVIMGLIGGSFFYGEVVITPAISVMSAIEGLEIVAPQLDTWIVPLSIIVLTLLFMIQKHGTAMVGKLFAPIMLTWFLILAGLGLRSIIANPEVLHALNPMWAVHFFLEYKTVSFIALGAVVLSITGVEALYADMGHFGKFPIRLAWFTVVLPSLTLNYFGQGALLLKNPEAIKNPFFLLAPDWALIPLLIIAALATVIASQAVISGVFSLTRQAVRLGYLSPMRIIHTSEMESGQIYIPFVNWMLYVAVVIVIVSFEHSSNLAAAYGIAVTGTMVLTSILSTTVARQNWHWNKYFVALILIAFLCVDIPLFTANLDKLLSGGWLPLSLGTVMFIVMTTWKSERFRLLRRMHEHGNSLEAMIASLEKSPPVRVPGTAVYMSRAINVIPFALMHNLKHNKVLHERVILLTLRTEDAPYVHNVRRVQIEQLSPTFWRVVASYGWRETPNVEEVFHRCGLEGLSCRMMETSFFMSHESLILGKRPWYLRLRGKLYLLLQRNALRAPDQFEIPPNRVIELGTQVEI

SEQ ID NO: 6 - protein, 549 aa, Escherichia coli
MLNQKIQNPNPDELMIEVDLCYELDPYELKLDEMIEAEPEPEMIEGLPASDALTPADRYLELFEHVQSAKIFPDSKTFPDCAPKMDPLDILIRYRKVRRHRDFDLRKFVENHFWLPEVYSSEYVSDPQNSLKEHIDQLWPVLTREPQDHIPWSSLLALPQSYIVPGGRFSETYYWDSYFTMLGLAESGREDLLKCMADNFAWMIENYGHIPNGNRTYYLSRSQPPVFALMVELFEEDGVRGARRYLDHLKMEYAFWMDGAESLIPNQAYRHVVRMPDGSLLNRYWDDRDTPRDESWLEDVETAKHSGRPPNEVYRDLRAGAASGWDYSSRWLRDTGRLASIRTTQFIPIDLNAFLFKLESAIANISALKGEKETEALFRQKASARRDAVNRYLWDDENGIYRDYDWRREQLALFSAAAIVPLYVGMANHEQADRLANAVRSRLLTPGGILASEYETGEQWDKPNGWAPLQWMAIQGFKMYGDDLLGDEIARSWLKTVNQFYLEQHKLIEKYHIADGVPREGGGGEYPLQDGFGWTNGVVRRLIGLYGEP

SEQ ID NO: 7 - protein, 412 aa, Escherichia coli
MKIYLVGGAVRDALLGLPVKDRDWVVVGSTPQEMLDAGYQQVGRDFPVFLHPQTHEEYALARTERKSGSGYTGFTCYAAPDVTLEDDLKRRDLTINALAQDDNGEIIDPYNGLGDLQNRLLRHVSPAFGEDPLRVLRVARFAARYAHLGFRIADETLALMREMTHAGELEHLTPERVWKETESALTTRNPQVFFQVLRDCGALRVLFPEIDALFGVPAPAKWHPEIDTGIHTLMTLSMAAMLSPQVDVRFATLCHDLGKGLTPPELWPRHHGHGPAGVKLVEQLCQRLRVPNEIRDLARLVAEFHDLIHTFPMLNPKTIVKLFDSIDAWRKPQRVEQLALTSEADVRGRTGFESADYPQGRWLREAWEVAQSVPTKAVVEAGFKGVEIREELTRRRIAAVASWKEQRCPKPE

SEQ ID NO: 8 - protein, 273 aa, Escherichia coli
MSDMHSLLIAAILGVVEGLTEFLPVSSTGHMIIVGHLLGFEGDTAKTFEVVIQLGSILAVVVMFWRRLFGLIGIHFGRPLQHEGESKGRLTLIHILLGMIPAVVLGLLFHDTIKSLFNPINVMYALVVGGLLLIAAECLKPKEPRAPGLDDMTYRQAFMIGCFQCLALWPGFSRSGATISGGMLMGVSRYAASEFSFLLAVPMMMGATALDLYKSWGFLTSGDIPMFAVGFITAFVVALIAIKTFLQLIKRISFIPFAIYRFIVAAAVYVVFF

SEQ ID NO: 9 - DNA, 449 bp, Escherichia coli
  source 1..449: mol_type = genomic DNA
gtaatagtgtgattaatcatatgcgtttttggttatgtgttgtttgacttaatatcagagccgagagatacttgttttctacaaaggagagggaaatgttgcaactaacaccacttcagttatttaaaaacctgtccgatgaaacccgtttgggtatcgtgttgttgctcagggagatgggagagttgtgcgtgtgtgatctttgcatggcactggatcaatcacagcccaaaatatcccgtcatctggcgatgctacgggaaagtggaatccttctggatcgtaaacagggaaaatgggttcactaccgcttatcaccgcatattccttcatgggctgcccagattattgagcaggcctggttaagccaacaggacgacgttcaggtcatcgcacgcaagctggcttcagttaactgctccggtagcagtaaggctgtctgcatctaa

SEQ ID NO: 10 - DNA, 469 bp, Staphylococcus aureus
  source 1..469: mol_type = genomic DNA
cttacttcttttattttcattcaaatatttgcttgcatgatgagtcgaaaatggttataatacactcaaataaatatttgaatgaagatgggatgataatatgaaaaaaaaagatacctgcgaaattttttgctacgatgaagaaaaagtgaaccgtattcagggagacttgcagaccgtggatatctccggtgtatcccagattctgaaggcgatcgcggatgaaaatcgtgccaagatcacttatgcgctgtgccaggatgaagaactttgtgtttgtgatattgcgaatattctcggggtgactatcgcgaatgcatcacaccacctgcgcacattgtacaaacaaggtgtggttaactttcgcaaagaaggtaaattggctctttattcgcttggagacgaacacatccgccaaattatgatgatcgcgctggcccataaaaaagaagtaaaagtgaacgtctaa

SEQ ID NO: 11 - DNA, 77 bp, Escherichia coli
  source 1..77: mol_type = genomic DNA
tcgcttattggcaaaatgacaattttgtcatttttctgtcaccggaaaatcagagcctggcgagtaaagttggcggc

SEQ ID NO: 12 - DNA, 172 bp, Escherichia coli
  source 1..172: mol_type = genomic DNA
gctgtttatcagtaactttgtctggctggggagccactatcgccgacgcttccgtgcggataacgcgattgctgcggcctgctactttgccggtcacttcctgatcgtccgctcgctgtatctctgataaaacttgactctggagtcgactccagagtgtatccttcggtta

SEQ ID NO: 13 - DNA, 495 bp, synthetic construct
  source 1..495: mol_type = other DNA
ttacggcatggcactacgcgccaagcccgcctcaccttgcagtgacgcaatcagagggcaagacacgtttcccttccgagcgtgacaagcgcacaccaattcagagagcaccgtctccatacgcgccaggtccgccattttctcacggacatctttcagtttgtgttccgcgaggcttgacgcctcttcacaatgggtgccgtcatccaaacgcagtaactctgcaatttcatccagcgaaaagcccagccgttgcgcgcttttcacgaatttaacacgcacgacgtcggcttcgccataacgccgaatcgagccgtatggcttatcaggctcccgcagcaaacctttccgctggtaaaaacggatcgtttctacgttcaccccagctgctttggcaaacaccccaatcgtcaggttttccaggttgttctccatatcgcttgactccgtacatgagtacggaagtaaggttacgctatccaatccaaattcaaa

SEQ ID NO: 14 - DNA, 177 bp, Escherichia coli
  source 1..177: mol_type = genomic DNA
aatcatgccatcttttatcagcgcttaccctgcgctgtaacacaaaggcttaagtttcaatgagtaaaaatgactcgctacccgcagcaggcgagtcatttttactcgtttatcatgccagattacccgtcatatcagcgtttcatcgttggcacggaagatgcaatacccgaagta

SEQ ID NO: 15 - protein, 238 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 16 - protein, 231 aa, synthetic construct
MSELIKENMHMKLYMEGTVNNHHFKCTSEGEGKPYEGTQTMRIKVVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTEMLYPADGGLEGRSDMALKLVGGGHLICNLKTTYRSKKPAKNLKMPGVYYVDRRLERIKEADKETYVEQHEVAVARYCDLPSKLGHK

SEQ ID NO: 17 - protein, 238 aa, synthetic construct
MSKGEELFTGVVPILVELDGDVNGHKFSVSGEGEGDATYGKLTLKFICTTGKLPVPWPTLVTTFSWGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 18 - protein, 239 aa, synthetic construct
MVSKGEELFTGVVPILVELDGDVNGHKFSVRGEGEGDATNGKLTLKLISTTGKLPVPWPTLVTTLGYGLMVFARYPDHMKQHDFFKSAMPEGYVQERTISFEDDGYYKTRAEVKFEGDTLVNRIVLKGIDFKEDGNILGHKLEYNFNSHNVYITADKQKNGIKANFKIRHNVEDGGVQLADHYQQNTPIGDGPVLLPDNHYLSYQSVLSKDPNEKRDHMVLKERVTAAGITHDMNELYK

SEQ ID NO: 19 - DNA, 354 bp, Escherichia coli
  source 1..354: mol_type = genomic DNA
atgttgcaactaacaccacttcagttatttaaaaacctgtccgatgaaacccgtttgggtatcgtgttgttgctcagggagatgggagagttgtgcgtgtgtgatctttgcatggcactggatcaatcacagcccaaaatatcccgtcatctggcgatgctacgggaaagtggaatccttctggatcgtaaacagggaaaatgggttcactaccgcttatcaccgcatattccttcatgggctgcccagattattgagcaggcctggttaagccaacaggacgacgttcaggtcatcgcacgcaagctggcttcagttaactgctccggtagcagtaaggctgtctgcatctaa

SEQ ID NO: 20 - DNA, 369 bp, Staphylococcus aureus
  source 1..369: mol_type = genomic DNA
atgaaaaaaaaagatacctgcgaaattttttgctacgatgaagaaaaagtgaaccgtattcagggagacttgcagaccgtggatatctccggtgtatcccagattctgaaggcgatcgcggatgaaaatcgtgccaagatcacttatgcgctgtgccaggatgaagaactttgtgtttgtgatattgcgaatattctcggggtgactatcgcgaatgcatcacaccacctgcgcacattgtacaaacaaggtgtggttaactttcgcaaagaaggtaaattggctctttattcgcttggagacgaacacatccgccaaattatgatgatcgcgctggcccataaaaaagaagtaaaagtgaacgtctaa

SEQ ID NO: 21 - DNA, 435 bp, synthetic construct
  source 1..435: mol_type = other DNA
ttacggcatggcactacgcgccaagcccgcctcaccttgcagtgacgcaatcagagggcaagacacgtttcccttccgagcgtgacaagcgcacaccaattcagagagcaccgtctccatacgcgccaggtccgccattttctcacggacatctttcagtttgtgttccgcgaggcttgacgcctcttcacaatgggtgccgtcatccaaacgcagtaactctgcaatttcatccagcgaaaagcccagccgttgcgcgcttttcacgaatttaacacgcacgacgtcggcttcgccataacgccgaatcgagccgtatggcttatcaggctcccgcagcaaacctttccgctggtaaaaacggatcgtttctacgttcaccccagctgctttggcaaacaccccaatcgtcaggttttccaggttgttctccat

(19) United States
(12) Patent Application Publication
(10) Pub. No.: US 2025/0137029 A1
(43) Pub. Date: May 1, 2025

(54) METHODS OF USE, SYSTEMS, AND BIOSENSING MICROBES

(71) Applicant: The Regents of the University of California, Oakland, CA (US)

(72) Inventors: Alyssa Jasmine Chiang, La Jolla, CA (US); Jeff Hasty, Encinitas, CA (US)

(21) Appl. No.: 18/837,354

(22) PCT Filed: Feb. 14, 2023
(86) PCT No.: PCT/US2023/062572

§ 371 Date: Aug. 9, 2024

Related U.S. Application Data
(60) Provisional application No. 63/309,882, filed on Feb. 14, 2022.

(51) Int. Cl. C12Q 1/02; C12N 15/70; G01N 21/64; G01N 33/18; G16B 40/20

(52) U.S. Cl. CPC C12Q 1/02; C12N 15/70; G01N 21/643; G01N 21/6456; G01N 33/18; G16B 40/20; C12N 2830/002

(57) ABSTRACT

Described herein are methods, compositions, and systems for determining an analyte in a sample. Method of determining an amount of an analyte in a sample using an adapted bio sensing microbe can include inducing an adapted bio sensing microbe containing an inducible promoter operably linked to a gene encoding a signal, wherein the inducible promoter is induced by the analyte to produce the signal; measuring the signal; and determining, in response to the signal, the amount of the analyte. The sample can include a high salinity medium (e.g., groundwater, seawater, road run-off water, anaerobic bioreactor water, and sewer water). The analyte can include heavy metal.

### CROSS-REFERENCE TO RELATED APPLICATION

This application claims priority to U.S. Provisional Patent Application No. 63/309,882, filed on Feb. 14, 2022. The disclosure of the prior application is considered part of the disclosure of this application, and is incorporated herein by reference in its entirety.

### FEDERALLY SPONSORED RESEARCH OR DEVELOPMENT

This invention was made with government support under GM069811 awarded by the National Institutes of Health and DGE1650112 awarded by the National Science Foundation. The government has certain rights in the invention.

### SEQUENCE LISTING

This application contains a Sequence Listing that has been submitted electronically as an ASCII text file named Sequence_Listing.xml. The ASCII text file, created on Feb. 14, 2023, is 26,472 bytes in size. The material in the ASCII text file is hereby incorporated by reference in its entirety.

### TECHNICAL FIELD

The present disclosure relates to methods, devices, systems, computer-implemented methods, and compositions that pertain to detecting an amount of analytes in a sample (e.g. heavy metals).

### BACKGROUND

Biosensors can enable the detection and/or quantification of environmental analytes, such as heavy metals, in a sample through the combination of a biological component (e.g. a bacterial strain) and a transducing device (e.g. a fluorescent reporter). However, many biosensing devices and/or systems need to function under stressful environments, like high salinity or extreme pH, which can be difficult. For example, high salinity can increase the background signal of biosensors, making detection and/or quantification of environmental analytes more difficult by decreasing sensitivity.

Furthermore, some biosensing systems use visualization devices such as microfluidic devices to detect the signal from the biosensor. Some live microorganisms, such as biosensing microbes or adapted biosensing microbes, when grown in a visualization device (e.g. a microfluidic device or microfluidic chamber) can form biofilms, or sticky masses of cells, that clog the microfluidic device. There is a clear need for biosensing microbes that function under stressful environments (e.g. a high salinity medium), maintain their biosensing sensitivity/ability, and/or do not interrupt (e.g., prevent the function of) the visualization device (e.g. clog a microfluidic device).

### SUMMARY

Provided herein are methods, composition, and systems using adapted biosensing microbes to detect an amount of an analyte in a sample (e.g., a heavy metal in a high salinity medium).

Described herein are methods of determining an amount of an analyte in a sample using an adapted biosensing microbe including inducing an adapted biosensing microbe in the sample, measuring the signal; and determining, in response to the signal, the amount of the analyte.

Also described herein are nucleic acid constructs including an inducible promoter operably linked to a gene encoding a signal, wherein the inducible promoter is induced by a threshold level of a heavy metal; a selectable marker; and an origin of replication.

Also described herein are methods of producing an adapted biosensing microbe to detect an analyte in a sample including obtaining a non-adapted biosensing microbe; growing the non-adapted biosensing microbe in a medium similar to the sample to generate one or more adapted biosensing microbes; and selecting the adapted biosensing microbe from the one or more adapted biosensing microbes by selecting for one or more traits selected from the group of increased growth rate compared to the non-adapted biosensing microbe, increased yield compared to the non-adapted biosensing microbe, increased sensitivity of detection of the analyte compared to the non-adapted biosensing microbe, increased production of the signal compared to the non-adapted biosensing microbe, decreased baseline of the signal in the absence of the analyte compared to the non-adapted biosensing microbe, decreased biofilm formation compared to the non-adapted biosensing microbe, increased synthesis of osmoprotectants compared to the non-adapted biosensing microbe, increased uptake of osmoprotectants compared to the non-adapted biosensing microbe, and increased resistance to toxicity associated with increased levels of osmoprotectants compared to the non-adapted biosensing microbe; thereby producing an adapted biosensing microbe to detect an analyte in a sample.

In any of the methods, compositions, or systems described herein, the sample includes a high salinity medium. In some cases, the sample is of a sample type selected from groundwater, seawater, road run-off water, anaerobic bioreactor water, and sewer water. In some cases, the analyte is a heavy metal. In some cases, the heavy metal is selected from arsenic, cadmium, copper, lead, mercury, and zinc.

In any of the methods, compositions, or systems described herein, the adapted biosensing microbe can include an inducible promoter operably linked to a gene encoding a signal, and the inducible promoter is induced by the analyte to produce the signal. In some cases, the adapted biosensing microbe also includes a modification in at least one of the genes selected from the group consisting of nagA, ompC, cueO, phoE, kup, treF, cca, and bacA, or homologous genes thereof, compared to the non-adapted microbe

In any of the methods, compositions, or systems described herein, the signal is selected from a fluorescence signal, a luminescence signal, and a colorimetric signal. In some cases, the signal is a fluorescence signal. In some cases, the signal is a fluorescence signal detected using an emission wavelength of 510 nm and excitation wavelength of 485 nm.

In some cases, the signal can be used to determine a feature selected from the group consisting of a fold-change response, a final signal measurement, a maximum signal measurement, a signal relaxation response, and a signal response rate.

In any of the methods, compositions, or systems described herein, the step of inducing also includes inducing the adapted biosensing microbe in a microfluidic device. In some cases, the step of inducing also includes imaging the microfluidic device at least once while inducing or after inducing the biosensing microbe to generate at least one image. In some cases, the step of inducing also includes imaging the microfluidic device at least twice while inducing or after inducing the biosensing microbe to generate at least two images. In some cases, any of the methods, compositions, or systems described herein can also include extracting growth data or the signal from at least one image or at least two images.

In any of the methods, compositions, or systems described herein, the step of determining can also include receiving, from a microbe-detecting sensor, the signal based on sensing the adapted biosensing microbes, and determining, in response to receiving the signal, the amount of the analyte. In some cases, any of the methods compositions, or systems described herein can also include at least one of the group selected from i) generating a report comprising the determine amount of the analyte; ii) storing data to a memory, the data comprising the determined amount of the analyte; iii) transmitting an alert, the alert comprising the determined amount of the analyte; and iv) initiating an automated device with automation instructions created based on the determined amount of the analyte. In some cases, determining the amount of the analyte includes submitting to an amount classifier, as input, the signal, wherein the amount classifier is created via machine learning techniques trained on training data comprising training signals and training amounts, and receiving, from the amount classifier as output, the amount of the analyte. In some cases, the machine learning techniques comprise at least one of the group selected from recurring neural networks, long short term memory, time series forest classifier, and shapelet-based classifier.

In any of the methods, compositions, or systems described herein, the inducible promoter is selected from the arsR promoter, the cadC promoter, the cusC promoter, the zntA promoter, the mer promoter and the zraP promoter. In some cases, the arsR promoter, the cusC promoter, and/or the zntA promoter is from an Escherichia coli (E. coli) strain. In some cases, the E. coli strain is E. coli R773 or E. coli MG1655. In some cases, the cadC promoter is from a Staphylococcus aureus (S. aureus) strain. In some cases, the S. aureus strain is S. aureus pl258. In some cases, the mer promoter is bidirectional. In some cases, the mer promoter is from a transposon. In some cases, the transposon is a Tn21 transposon.

In any of the methods, compositions, or systems described herein the selectable marker is an antibiotic selectable marker. In some cases, the antibiotic selectable marker confers resistance to an antibiotic or a toxin. In some cases, the antibiotic is selected from ampicillin, kanamycin, chloramphenicol, erythromycin, spectinomycin, neomycin, streptomycin, zeocin, and gentamicin.

In any of the methods, compositions, or systems described herein, the origin of replication is p15A.

In any of the methods, compositions, or systems described herein, the biosensing microbe (e.g., adapted biosensing microbe) can include any of the nucleic acid constructs described herein. In some cases, the microbe is selected from a fungi, a bacterium, an algae, a protozoan, and a nematode. In some cases, the fungi is of a genera selected from Saccharomyces and Pichia. In some cases, the fungi is of a species selected from Saccharomyces cerevisiae and Pichia pastoris. In some cases, the bacterium is of a genera selected from Escherichia, Streptococcus, Staphylococcus, Salmonella, Campylobacter, Pseudomonas, Bacillus, Klebsiella, and Vibrio. In some cases, the bacterium is from a species selected from Escherichia coli and Staphylococcus aureus.

In any of the methods, compositions, or systems described herein, the step of; growing the non-adapted biosensing microbe in a medium similar to the sample to generate one or more adapted biosensing microbes can also include serially-passaging the adapted biosensing microbe at least once to fresh medium to generate a further adapted biosensing microbe. In some cases, the fresh medium is the same type of medium as the medium.

Also disclosed here in are systems for use in determining an amount of an analyte in a sample using an adapted biosensing microbe. The system can include a microbe-detecting sensor configured to generate a signal based on sensing the adapted biosensing microbe in the sample; one or more processors; and memory storing instructions that, when executed by the processors, cause the processors to perform operations that can include receiving, from the microbe-detecting sensor, a signal based on sensing any of the adapted biosensing microbes described herein, and determining, in response to receiving the signal, the amount of the analyte.

In some cases, the operations also include at least one of the group selected from i) generating a report comprising the determine amount of the analyte; ii) storing data to the memory, the data comprising the determined amount of the analyte; iii) transmitting an alert, the alert comprising the determined amount of the analyte; and iv) initiating an automated device with automation instructions created based on the determined amount of the analyte.

In some cases, the determining the amount of the analyte includes submitting to an amount classifier, as input, the signal, wherein the amount classifier is created via machine learning techniques trained on training data comprising training signals and training amounts, and receiving, from the amount classifier as output, the amount of the analyte. In some cases, the machine learning techniques include at least one of the group selected from recurring neural networks, long short term memory, time series forest classifier, and shapelet-based classifier.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A-1K show generation of adapted biosensing Escherichia coli strains. FIG. 1A is an exemplary schematic of a general protocol to evolve and transform strains. FIG. 1B is a series of graphs depicting growth curves of adapted strains grown in HM9 minimal media and HM9 minimal media+50% seawater. FIG. 1C is a graph comparing growth rates of the ancestral E. coli strain MG1655 and adapted E. coli strains R1-R6 in HM9 minimal media (HM9) and HM9+50% seawater. FIG. 1D is a series of graphs showing that adapted E. coli strains were more robust in increased salt concentrations (NaCl in HM9) than the ancestral E. coli strain MG1655. FIG. 1E is an exemplary heavy metal sensing plasmid with optional transcription factor, promoter (left-facing arrow), and one or more terminators (right-hand T), and schematic of adapted strain transformation to generate 36 total E. coli biosensing strains. FIGS. 1F-1K depict a series of exemplary heavy metal sensing plasmid maps for arsenic (FIG. 1F), mercury (FIG. 1G), copper (FIG. 1H), cadmium (FIG. 1I), zinc (FIG. 1J), and lead (FIG. 1K). FIG. 1L is an image of an exemplary microfluidic device experiment comparing growth of Hg3_Mg1655 (Hg_MG) and Hg3_R1 (Hg_R1). Hg_MG formed more biofilm that fluoresced, likely as a result of stress. No inducer was added—the image shown reflects growth on 50% seawater with a background of HM9 minimal media.

FIGS. 2A-2N show responses of adapted biosensing E. coli strain to heavy metals. FIG. 2A shows an exemplary spotting region on a microfluidic chip. FIG. 2B shows an exemplary microfluidic chip. FIG. 2C shows an exemplary loading pattern of microfluidic chip with strains R1-R6 for each heavy metal analysis. FIG. 2D is a time-series graph depicting induction of all 6 evolved strains containing the mercury-sensing plasmid with 1 ppm mercury, including the time period prior to addition of mercury to the HM9+50% seawater media, the 4-hour induction period, and the time period after mercury was removed and the media was replaced with fresh HM9+50% seawater. FIG. 2E is a graph of the time derivative of FIG. 2D, which illustrates the rate of response throughout mercury induction.

FIG. 2F depicts two exemplary images of the filled cell traps in the microfluidics experiment with mercury induction.

FIG. 2G is a graph of a time-series graph depicting induction of all 6 evolved strains containing the lead-sensing plasmid with 1 ppm lead, including the time period prior to addition of lead to the HM9+50% seawater media, the 4-hour induction period, and the time period after lead was removed and the media was replaced with fresh HM9+50% seawater. FIG. 2H is a graph of the time derivative of FIG. 2G, which illustrates the rate of response throughout lead induction.

FIG. 2I is a graph of a time-series graph depicting induction of all 6 evolved strains containing the arsenic-sensing plasmid with 1 ppm arsenic, including the time period prior to addition of arsenic to the HM9+50% seawater media, the 4-hour induction period, and the time period after arsenic was removed and the media was replaced with fresh HM9+50% seawater. FIG. 2J is a graph of time derivative of FIG. 2I, which illustrates the rate of response throughout lead induction.

FIG. 2K is a graph of a time-series graph depicting induction of all 6 evolved strains containing the cadmium-sensing plasmid with 1 ppm cadmium, including the time period prior to addition of cadmium to the HM9+50% seawater media, the 4-hour induction period, and the time period after cadmium was removed and the media was replaced with fresh HM9+50% seawater. FIG. 2L is a graph of time derivative of FIG. 2I, which illustrates the rate of response throughout cadmium induction.

FIG. 2M is a graph of time-series graph depicting induction of all 6 evolved strains containing the copper-sensing plasmid with 1 ppm copper, including the time period prior to addition of copper to the HM9+50% seawater media, the 4-hour induction period, and the time period after copper was removed and the media was replaced with fresh HM9+50% seawater. FIG. 2N is a graph of time derivative of FIG. 2I, which illustrates the rate of response throughout copper induction.

FIGS. 3A-3K show an exemplary process of identifying more sensitive adapted biosensing E. coli strains. FIGS. 3A-3B are exemplary graphs of GFP expression following exposure to 1 ppm mercury (Hg) (FIG. 3A), and 5 ppm Hg (FIG. 3B) of E. coli strains Hg3_R1 (R1), Hg3_R2 (R2), Hg3_R3 (R3), Hg4_R4 (R4), Hg5_R5 (R5), Hg6_R6 (R6), and non-adapted E. coli strain MG1655. FIG. 3C depicts the fold change of a response to 1 ppm Hg (4×) or 5 ppm Hg (20×) of E. coli strains Hg3_R1 (R1), Hg3_R2 (R2), Hg3_R3 (R3), Hg4_R4 (R4), Hg5_R5 (R5), Hg6_R6 (R6), and non-adapted E. coli strain MG1655. FIG. 3D is a series of exemplary graphs of GFP expression following exposure to 0.5 ppm lead (Pb) (FIG. 3D—left), 1 ppm Pb (FIG. 3D—middle), and 5 ppm Pb (FIG. 3D—right) of E. coli strains Pb7_R1 (R1), Pb7_R2 (R2), Pb7_R3 (R3), Pb7_R4 (R4), Pb7_R5 (R5), Pb7_R6 (R6), and non-adapted E. coli strain MG1655. FIG. 3E depicts the fold change of a response to 0.5 ppm Pb (2×), 1 ppm Pb (4×), or 5 ppm Pb (20×) of E. coli strains Pb7_R1 (R1), Pb7_R2 (R2), Pb7_R3 (R3), Pb7_R4 (R4), Pb7_R5 (R5), Pb7_R6 (R6), and non-adapted E. coli strain MG1655. FIG. 3F is a series of exemplary graphs of GFP expression following exposure to 0.5 ppm arsenic (As) (FIG. 3F—left), 1 ppm As (FIG. 3F—middle), and 5 ppm As (FIG. 3F—right) of E. coli strains As7_R1 (R1), As7_R2 (R2), As7_R3 (R3), As7_R4 (R4), As7_R5 (R5), As7_R6 (R6), and non-adapted E. coli strain MG1655. FIG. 3G depicts the fold change of a response to 0.5 ppm As (2×), 1 ppm As (4×), or 5 ppm As (20×) of E. coli strains As7_R1 (R1), As7_R2 (R2), As7_R3 (R3), As7_R4 (R4), As7_R5 (R5), As7_R6 (R6), and non-adapted E. coli strain MG1655. FIG. 3H is a series of exemplary graphs of GFP expression following exposure to 0.5 ppm cadmium (Cd) (FIG. 3H—left), 1 ppm Cd (FIG. 3H—middle), and 5 ppm Cd (FIG. 3H—right) of E. coli strains Cd1_R1 (R1), Cd1_R2 (R2), Cd1_R3 (R3), Cd1_R4 (R4), Cd1_R5 (R5), Cd1_R6 (R6), and non-adapted E. coli strain MG1655. FIG. 3I depicts the fold change of a response to 0.5 ppm Cd (2×), 1 ppm Cd (4×), or 5 ppm Cd (20×) of E. coli strains Cd1_R1 (R1), Cd1_R2 (R2), Cd1_R3 (R3), Cd1_R4 (R4), Cd1_R5 (R5), Cd1_R6 (R6), and non-adapted E. coli strain MG1655. FIG. 3J is a series of exemplary graphs of GFP expression following exposure to 0.5 ppm copper (Cu) (FIG. 3J—left), 1 ppm Cu (FIG. 3J—middle), and 5 ppm Cu (FIG. 3J—right) of E. coli strains Cu3_R1 (R1), Cu3_R2 (R2), Cu3_R3 (R3), Cu3_R4 (R4), Cu3_R5 (R5), Cu3_R6 (R6), and non-adapted E. coli strain MG1655. FIG. 3K depicts the fold change of a response to 0.5 ppm Cu (2×), 1 ppm Cu (4×), or 5 ppm Cu (20×) of E. coli strains Cu3_R1 (R1), Cu3_R2 (R2), Cu3_R3 (R3), Cu3_R4 (R4), Cu3_R5 (R5), Cu3_R6 (R6), and non-adapted E. coli strain MG1655. Black boxes around the strain names in any of FIGS. 3A-3K indicate exemplary heavy metal-responding adapted biosensing E. coli strains, that exhibit a heavy-metal dose-dependent increase in the fold-change of GFP induction.

FIGS. 4A-4I show how to determine the identity, and optionally the concentration of a heavy metal in an unknown sample, with a combinatorial approach. FIG. 4A is an exemplary schematic of generating a training data set for a machine learning algorithm, where training data can use one or more physiological measures determined by experiments such as those described in FIGS. 4B and 4C. Exemplary physiological measures can include GFP fold-change response, rate of response (first derivative), or response relaxation behavior post-induction of an adapted biosensing E. coli strain when exposed to an unknown sample compared to the same strain when exposed to a known heavy metal of a known concentration. FIGS. 4D, 4F, and 4H are a series of induction curves of selected adapted biosensing E. coli strains responding to a gradient of corresponding mercury, arsenic, and copper inductions, respectively, with L1 and L2 being equivalent “low” concentrations, M1 and M2 being equivalent “medium” concentrations, and H1 and H2 being equivalent “high” concentrations, all as outlined in FIG. 4C. FIGS. 4E, 4G, and 4I are graphs of the GFP fold induction as computed from FIGS. 4D, 4F, and 4H.

FIG. 5 shows an example process that can be used to produce classifiers able to evaluate the signal based on sensing the adapted biosensing microbes, and determining, in response to receiving the signal, the amount of the analyte.

FIG. 6 shows an example process for determining the amount of an analyte in a sample.

FIG. 7 shows an example of a computing device.

FIG. 8 shows an exemplary schematic of serial dilutions.

### DETAILED DESCRIPTION

This disclosure describes compositions, systems, and methods of use thereof for determining an amount of an analyte in a sample using an adapted biosensing microbe. In practice, adapted biosensing microbes can be evolved (adapted) or genetically engineered to improve their ability to determine an amount of an analyte (e.g., a heavy metal). In some cases, the analyte (e.g., heavy metal) is in a high salinity medium.

### Methods of Determining an Amount of An Analyte in A Sample

Methods of determining an amount of an analyte in a sample using an adapted biosensing microbe can include inducing an adapted biosensing microbe in the sample, measuring the signal, and determining, in response to the signal, the amount of the analyte. In some cases, the adapted biosensing microbe can include an inducible promoter operably linked to a gene encoding a signal. In some cases, the inducible promoter is induced by the analyte to produce the signal. In some cases, an adapted biosensing microbe can determine an amount of an analyte (e.g., a heavy metal such as arsenic, cadmium, copper, lead, mercury, or zinc) in a sample of a high salinity medium.

An analyte can include any component of a sample. In some cases, an analyte is a chemical. In some cases, an analyte is a heavy metal. A heavy metal is a metal with a density greater than about 5 g/cm³. Heavy metals can include manganese, cobalt, nickel, selenium, silver, tin, antimony, thallium, arsenic, cadmium, copper, lead, mercury, and zinc. In some cases, the heavy metal is selected from the group consisting of arsenic, cadmium, copper, lead, mercury, and zinc. In some cases, the heavy metal is selected from the group consisting of arsenic, cadmium, copper, lead, and mercury.

An amount of an analyte can include the presence or absence of the analyte in a sample. If the analyte is present in the sample, the amount of the analyte in the sample can be determined. For example, the amount of the analyte can include the concentration of the analyte (e.g., micrograms per liter or parts per million).

A microbe in high salinity media, or high osmolarity media, attempt to maintain internal osmotic pressure, which depends on the internal osmolarity relative to the external osmolarity. This can be considered maintaining homeostasis. In a high salinity medium, water exits the microbial cell via osmosis, which causes cytoplasmic volume, and therefore the cell volume, to decrease. In addition, the microbial cell can accumulate small molecular weight solutes including ions such as potassium (K⁺), glutamate, trehalose, and glycine-betaine. These responses to a high salinity medium can result in a decreased sensitivity to heavy metals, an increased baseline of heavy metal sensing, or increased growth stress, which can result in a slower growth rates compared to growth rates of a microbe in a lower-salinity medium.

In addition, a microbe in high salinity media, or high osmolarity media, can produce osmoprotectants. Osmoprotectants include small organic molecules with neutral charge and low toxicity at high concentrations that act as osmolytes and help microbes survive extreme osmotic stress. Osmoprotectants can be placed in three chemical classes: betaines and associated molecules, sugars and polyols, and amino acids. These molecules accumulate in the microbial cell and balance the osmotic difference between the microbe's external environment and the microbe's cytosol. (See, for example, Wood, J M. Annual review of microbiology 65 (2011): 215-238.).

As used herein, a sample can include any liquid of which an amount of an analyte can be determined. For example, a sample can include a high salinity medium. As described herein, a “high salinity medium” can include any medium with more salt in the sample compared to a sample of fresh water. For example, brackish water, water from estuaries, groundwater, seawater, road run-off water, and sewer water. In some cases, the sample is of a sample type selected from groundwater, seawater, road run-off water, bioreactor water, and sewer water. In some cases, bioreactor water can include anaerobic bioreactor water. In some cases, the sample is of a sample type selected from groundwater, seawater, road run-off water, anaerobic bioreactor water, and sewer water.

Salinity of a sample can be defined using any standard salinity measurement. Non-limiting examples of salinity measurements include parts of salt per thousand (ppt), percent of weight salt per weight of the sample (% wt/wt), concentration of salt (e.g., molarity, such as miillimolarity, grams of salt per liter of sample (g/L) orgrams of salt per kilogram of sample (g/kg)), or electrical conductivity of the sample (EC/w). Salinity can measure a specific salt, such as sodium chloride (NaCl), or at least one other type of salt (e.g., NaCl and at least one other salt). Non-limiting examples of other salts than can contribute to salinity include magnesium sulfate, potassium nitrate, and sodium bicarbonate. Salinity can be measured using any standard salinity measurement device. Non-limiting examples of salinity measurement devices can include a refractometer, a hydrometer, and a conductivity meter.

A high salinity medium can also include any liquid with at least 0.5 ppt of salt. For example, a high salinity medium can include any liquid with between about 0.5 ppt to about 100 ppt of salt (e.g., between about 0.5 ppt to about 75 ppt, between about 0.5 ppt to about 50 ppt, between about 0.5 ppt to about 40 ppt, between about 0.5 ppt to about 30 ppt, between about 0.5 ppt to about 25 ppt, between about 1 ppt to about 100 ppt, between about 5 ppt to about 100 ppt, between about 25 ppt to about 100 ppt, between about 30 ppt to about 100 ppt, between about 40 ppt to about 100 ppt, between about 50 ppt to about 100 ppm, or between about 6 ppt to about 50 ppt). Salinity can also be measure as a percentage of salt per weight of the liquid. A high salinity liquid medium can include any liquid with greater than 0.5% salt (e.g., between about 0.5% salt to about 10% salt, between about 1% salt to about 10% salt, or between about 2% salt to about 7% salt). A high salinity medium can also include any liquid with at least 8.5 mM. For example, a high salinity medium can include any liquid with between about 8.5 mM to about 1800 mM (e.g., between about 8.5 mM to about 1500 mM, between about 8.5 mM to about 1000 mM, between about 8.5 mM to about 800 mM, between about 8.5 mM to about 500 mM, between about 10 mM to about 1800 mM, between about 50 mM to about 1800 mM, between about 100 mM to about 1800 mM, between about 500 mM to about 1800 mM, between about 100 mM to about 1500 mM, or between about 200 mM to about 800 mM).

Inducing an adapted biosensing microbe in a sample can include exposing the adapted biosensing microbe to the sample that is being tested for the analyte (e.g., heavy metals. Inducing the adapted biosensing microbe by exposing the adapted biosensing microbe to the sample can occur over various time frames ranging from seconds to hours. For example, induction can occur over a time frame of seconds, minutes, hours, or days. For example, an adapted biosensing microbe can be induced for about 30 minutes to about 6 hours (e.g., about 1 hour, about 2 hours, or about 3 hours).

Measuring the signal (e.g., the signal produced by an adapted biosensing microbe, also referred to as 604 sensing the biosensing microbe signal), can use any appropriate 304 microbe detecting sensor. Exemplary microbe detecting sensors can include, but are not limited to, fluorescence sensors, luminescence sensors, or colorimetric sensors. For example, microscopes, plate readers (E.g., TECAN™ plate readers), or microfluidic devices with a means for fluorescence sensing, luminescence sensing, or colorimetric sensing can be used. Means for sensing fluorescence signals or luminescence signals include sensors designed to emit and collect specific wavelengths of light that are produced by the biosensing microbe (e.g., the adapted biosensing microbe). Sensing fluorescence signals or luminescence signals can use sensors or cameras. Exemplary microfluidic devices are described in U.S. Pat. No. 11,209,412, issued Dec. 28, 2021, which is hereby incorporated in its entirety.

In some cases, inducing the biosensing microbe (e.g., the adapted biosensing microbe) occurs in a microfluidic device. In some cases, inducing the biosensing microbe (e.g., the adapted biosensing microbe) can also include imaging the microfluidic device while inducing the biosensing microbe to generate at least one image. In some cases, inducing the biosensing microbe (e.g., the adapted biosensing microbe) can also include imaging the microfluidic device at least once (e.g., once, twice, three times, etc.) while inducing the biosensing microbe to generate at least one image. In some cases, inducing the biosensing microbe (e.g., the adapted biosensing microbe) can also include imaging the microfluidic device once while inducing the biosensing microbe to generate at least one image. In some cases, inducing the biosensing microbe (e.g., the adapted biosensing microbe) can also include imaging the microfluidic device twice while inducing the biosensing microbe to generate at least one image.

The signal can be analyzed to produce various features. Features can include the fold-change response of the signal (ratio of the maximal response upon induction to the basal signal level in the absence of inducer), the signal relaxation response (the behavior of the signal post-induction), and the signal response rate (the slope of the signal during induction, determined by calculating the first derivative of a graph of the signal overtime), the maximum signal measurement, and a final signal measurement. In some cases, the signal can be used to determine a feature selected from the group consisting of a fold-change response, a final signal measurement, a maximum signal measurement, a signal relaxation response, and a signal response rate.

Determining, in response to the signal, the amount of the substance, can include using any of the systems and algorithms described herein. In some cases, determining the amount of the substance can include extracting growth data or the signal from at least one image or at least two images taken during induction of the biosensing microbe (e.g., the adapted biosensing microbe) in a microfluidic device. In some cases, determining, in response to the signal, the amount of the substance, can include 606 receiving, from a microbe-detecting sensor, the signal based on sensing the adapted biosensing microbes, and 626 determining, in response to receiving the signal, the amount of the substance.

In some cases, any of the methods described herein can also include at least one of the group selected from i) generating a report comprising the determine amount of the substance; ii) storing data to a memory, the data comprising the determined amount of the substance; iii) transmitting an alert, the alert comprising the determined amount of the substance; and iv) initiating an automated device with automation instructions created based on the determined amount of the substance.

In some cases, determining the amount of the substance also includes, submitting to an amount classifier, as input, the signal, wherein the amount classifier is created via machine learning techniques trained on training data comprising training signals and training amounts, and receiving, from the amount classifier as output, the amount of the substance. In some cases, the machine learning techniques comprise at least one of the group selected from recurring neural networks, long short term memory, time series forest classifier, and shapelet-based classifier.

### Adapted Biosensing Microbes

A microbial population with favorable or desirable traits can be obtained via adaptation (e.g., adaptive laboratory evolution). Adaptive laboratory evolution is an approach wherein the process of natural selection is mimicked to evolve proteins or nucleic acids towards a user-defined goal. Laboratory adaptation can introduce modifications at the transcriptional, translational, and post-translational levels. The transcriptional level includes changes at the promoter (such as changing sigma factor affinity or binding sites for transcription factors), changing transcription terminators and attenuators, or non-synonymous mutations that alter the amino acid sequence (e.g., point mutations). The translational level includes changes at the ribosome binding sites and changing mRNA degradation signals. The post-translational level includes mutating an enzyme's active site and changing protein-protein interactions. These changes can be achieved in a multitude of ways. Reduction of expression level (or complete abolishment) can be achieved by swapping the native ribosome binding site (RBS) or promoter with another with lower strength/efficiency. ATG start sites can be swapped to a alternative start sites, such as a GTG, TTG, or CTG start codon, which results in reduction in translational activity of the coding region. Complete abolishment of expression can be done by knocking out (deleting) the coding region of a gene. Frameshifting the open reading frame (ORF) (e.g., by adding or deleting nucleotide positions in the nucleic acid) likely will result in a premature stop codon along the ORF, thereby creating a less-functional or non-functional truncated product. Insertion of in-frame stop codons will also similarly create a non-functional truncated product.

Adapted microbes (e.g., adapted biosensing microbes) can include one or more traits that were selected for during the adaptation process (see, “Methods of Producing Adapted Biosensing Microbes” section for more details about the adaptation process). Described herein are microbes adapted to growth in high salinity medium. In some cases, the one or more selected traits can be selected from the group of increased growth rate compared to the non-adapted biosensing microbe, increased yield compared to the non-adapted biosensing microbe, increased sensitivity of detection of the analyte compared to the non-adapted biosensing microbe, increased production of the signal compared to the non-adapted biosensing microbe, decreased baseline of the signal in the absence of the analyte compared to the non-adapted biosensing microbe, decreased biofilm formation compared to the non-adapted biosensing microbe, increased synthesis of osmoprotectants compared to the non-adapted biosensing microbe, increased uptake of osmoprotectants compared to the non-adapted biosensing microbe, and increased resistance to toxicity associated with increased levels of osmoprotectants compared to the non-adapted biosensing microbe.

In some cases, the adapted microbe and/or the adapted biosensing microbe has one or more modifications in at least one of the genes selected from the group consisting of nagA, ompC, cueO, phoE, kup, treF, cca, bacA, and homologous genes thereof, compared to the non-adapted microbe. The one more modifications can results in any of the selected traits described herein. Exemplary amino acid sequences of NagA, OmpC, CueO, PhoE, Kup, TreF, Cca, and BacA are in Table 1.

The nagA gene encodes a protein involved in the N-acetylglucosamine catabolic operon, which is a part of peptidoglycan and therefore a part of the cell wall. NagA converts N-acetylglucosamine-6-phosphate (GlcNAc-6-P) to glucosamine-6-phosphate (GlcN-6-P) using a deacetylase reaction. GlcN-6-P can enter either the glycolysis pathway or a pathway for the biosynthesis for liposaccharides. In some cases, a modification in nagA in the adapted biosensing microbe is present. In some cases, a modification in nagA in the adapted biosensing microbe results in lower final yield or growth saturation compared to the non-adapted microbe. In some cases, the adapted biosensing microbe includes a nagA gene with a nucleic acid sequence that is different from the nucleic acid sequence of the nagA gene of the non-adapted microbe.

The ompC gene encodes for outer membrame porin E, which is associated with high salinity and encodes a protein that generates a pore for small hydrophilic molecules to enter the periplasmic space of a microbial cell. envZ/ompR are a response regulator pair. OmpR induces ompC and ompF expression reciprocally under high and low salinity conditions. In some cases, a modification in ompC in the adapted biosensing microbe is present compared to the non-adapted microbe. In some cases, the adapted biosensing microbe includes an ompC gene with a nucleic acid sequence that is different from the nucleic acid sequence of the ompC gene of the non-adapted microbe.

The cueO gene encodes a periplasmic copper oxidase. Elevated levels of Cu(I) in periplasmic space are correlated with osmotic stress. As Cu(I) is cytotoxic at high levels, when the periplasmic copper oxidase of the cueO gene oxidizes Cu(I) to Cu(II), a less toxic analyte is formed. In some cases, a modification in cueO in the adapted biosensing microbe is present compared to the non-adapted microbe. In some cases, the adapted biosensing microbe includes a cueO gene with a nucleic acid sequence that is different from the nucleic acid sequence of the cueO gene of the non-adapted microbe.

The phoE gene encodes a porin. The PhoE porin can be induced by phosphate deprivation. Decreased expression of phoE is found in cells adapted to high osmolarity. In some cases, a modification in phoE in the adapted biosensing microbe is present compared to the non-adapted microbe. In some cases, the adapted biosensing microbe includes a phoE gene with a nucleic acid sequence that is different from the nucleic acid sequence of the phoE gene of the non-adapted microbe.

The kup gene is a component of the potassium uptake system in E. coli. Potassium is an analyte that E. coli can accumulate under osmotic stress. In some cases, a modification in kup in the adapted biosensing microbe is present compared to the non-adapted microbe. In some cases, the adapted biosensing microbe includes a kup gene with a nucleic acid sequence that is different from the nucleic acid sequence of the kup gene of the non-adapted microbe.

The treF gene encodes an enzyme called trehalase, which catalyzes hydrolysis of trehalose. Trehalose is an analyte that E. coli can accumulate under osmotic stress. Increased expression of treF is associated with high salinity conditions. In some cases, a modification in treF in the adapted biosensing microbe is present compared to the non-adapted microbe. In some cases, the adapted biosensing microbe includes a treFgene with a nucleic acid sequence that is different from the nucleic acid sequence of the treF gene of the non-adapted microbe.

The cca gene encodes the enzyme tRNA nucleotidyltransferase that catalyzes a cytidine-cytidine-adenosine (CCA) addition to tRNA to form a tRNA-NCCA product, which is a component of producing mature tRNAs. In some cases, a modification in cca in the adapted biosensing microbe is present compared to the non-adapted microbe. In some cases, the adapted biosensing microbe includes a cc gene with a nucleic acid sequence that is different from the nucleic acid sequence of the cca gene of the non-adapted microbe.

The bacA gene (also referred to as uppP) encodes a hydrophobic membrane protein that can have undecaprenyl pyrophosphate phosphatase activity. In some cases, a modification in bacA in the adapted biosensing microbe is present compared to the non-adapted microbe. In some cases, the adapted biosensing microbe includes a bacA gene with a nucleic acid sequence that is different from the nucleic acid sequence of the bacA gene of the non-adapted microbe.

TABLE 1
Exemplary amino acid sequences.

| SEQ | | |
|---|---|---|
| ID | | |
| NO: | Protein | Amino Acid Sequence |
| 1 | NagA | MYALTQGRIFTGHEFLDDHAVVIADGLIKSVCPVAELPPEIEQRSLNGAILSPGFIDV |
| | | QLNGCGGVQFNDTAEAVSVETLEIMQKANEKSGCTNYLPTLITTSDELMKQGVR |
| | | VMREYLAKHPNQALGLHLEGPWLNLVKKGTHNPNFVRKPDAALVDFLCENADVI |
| | | TKVTLAPEMVPAEVISKLANAGIVVSAGHSNATLKEAKAGFRAGITFATHLYNAMP |
| | | YITGREPGLAGAILDEADIYCGIIADGLHVDYANIRNAKRLKGDKLCLVTDATAPAG |
| | | ANIEQFIFAGKTIYYRNGLCVDENGTLSGSSLTMIEGVRNLVEHCGIALDEVLRMAT |
| | | LYPARAIGVEKRLGTLAAGKVANLTAFTPDFKITKTIVNGNEVVTQ |
| 2 | OmpC | MKVKVLSLLVPALLVAGAANAAEVYNKDGNKLDLYGKVDGLHYFSDNKDVDGD |
| | | QTYMRLGFKGETQVTDQLTGYGQWEYQIQGNSAENENNSWTRVAFAGLKFQD |
| | | VGSFDYGRNYGVVYDVTSWTDVLPEFGGDTYGSDNFMQQRGNGFATYRNTDFF |
| | | GLVDGLNFAVQYQGKNGNPSGEGFTSGVTNNGRDALRQNGDGVGGSITYDYEG |
| | | FGIGGAISSSKRTDAQNTAAYIGNGDRAETYTGGLKYDANNIYLAAQYTQTYNATR |
| | | VGSLGWANKAQNFEAVAQYQFDFGLRPSLAYLQSKGKNLGRGYDDEDILKYVDV |
| | | GATYYFNKNMSTYVDYKINLLDDNQFTRDAGINTDNIVALGLVYQF |
| 3 | CueO | MQRRDFLKYSVALGVASALPLWSRAVFAAERPTLPIPDLLTTDARNRIQLTIGAGQ |
| | | STFGGKTATTWGYNGNLLGPAVKLQRGKAVTVDIYNQLTEETTLHWHGLEVPGE |
| | | VDGGPQGIIPPGGKRSVTLNVDQPAATCWFHPHQHGKTGRQVAMGLAGLVVIE |
| | | DDEILKLMLPKQWGIDDVPVIVQDKKFSADGQIDYQLDVMTAAVGWFGDTLLT |
| | | NGAIYPQHAAPRGWLRLRLLNGCNARSLNFATSDNRPLYVIASDGGLLPEPVKVS |
| | | ELPVLMGERFEVLVEVNDNKPFDLVTLPVSQMGMAIAPFDKPHPVMRIQPIAISA |
| | | SGALPDTLSSLPALPSLEGLTVRKLQLSMDPMLDMMGMQMLMEKYGDQAMA |
| | | GMDHSQMMGHMGHGNMNHMNHGGKFDFHHANKINGQAFDMNKPMFAA |
| | | AKGQYERWVISGVGDMMLHPFHIHGTQFRILSENGKPPAAHRAGWKDTVKVEG |
| | | NVSEVLVKFNHDAPKEHAYMAHCHLLEHEDTGMMLGFTV |
| 4 | PhoE | MKKSTLALVVMGIVASASVQAAEIYNKDGNKLDVYGKVKAMHYMSDNASKDGD |
| | | QSYIRFGFKGETQINDQLTGYGRWEAEFAGNKAESDTAQQKTRLAFAGLKYKDLG |
| | | SFDYGRNLGALYDVEAWTDMFPEFGGDSSAQTDNFMTKRASGLATYRNTDFFG |
| | | VIDGLNLTLQYQGKNENRDVKKQNGDGFGTSLTYDFGGSDFAISGAYTNSDRTNE |
| | | QNLQSRGTGKRAEAWATGLKYDANNIYLATFYSETRKMTPITGGFANKTQNFEA |
| | | VAQYQFDFGLRPSLGYVLSKGKDIEGIGDEDLVNYIDVGATYYFNKNMSAFVDYKI |
| | | NOLDSDNKLNINNDDIVAVGMTYQF |
| 5 | Kup | MSTDNKQSLPAITLAAIGVVYGDIGTSPLYTLRECLSGQFGFGVERDAVFGFLSLIF |
| | | WLLIFVVSIKYLTFVMRADNAGEGGILTLMSLAGRNTSARTTSMLVIMGLIGGSFF |
| | | YGEVVITPAISVMSAIEGLEIVAPQLDTWIVPLSIIVLTLLFMIQKHGTAMVGKLFAP |
| | | IMLTWFLILAGLGLRSIIANPEVLHALNPMWAVHFFLEYKTVSFIALGAVVLSITGVE |
| | | ALYADMGHFGKFPIRLAWFTVVLPSLTLNYFGQGALLLKNPEAIKNPFFLLAPDWA |
| | | LIPLLIIAALATVIASQAVISGVFSLTRQAVRLGYLSPMRIIHTSEMESGQIYIPFVN |
| | | WMLYVAVVIVIVSFEHSSNLAAAYGIAVTGTMVLTSILSTTVARQNWHWNKYFVALI |
| | | LIAFLCVDIPLFTANLDKLLSGGWLPLSLGTVMFIVMTTWKSERFRLLRRMHEHGN |
| | | SLEAMIASLEKSPPVRVPGTAVYMSRAINVIPFALMHNLKHNKVLHERVILLTLRTE |
| | | DAPYVHNVRRVQIEQLSPTFWRVVASYGWRETPNVEEVFHRCGLEGLSCRMME |
| | | TSFFMSHESLILGKRPWYLRLRGKLYLLLQRNALRAPDQFEIPPNRVIELGTQVEI |
| 6 | TreF | MLNQKIQNPNPDELMIEVDLCYELDPYELKLDEMIEAEPEPEMIEGLPASDALTPA |
| | | DRYLELFEHVQSAKIFPDSKTFPDCAPKMDPLDILIRYRKVRRHRDFDLRKFVENHF |
| | | WLPEVYSSEYVSDPQNSLKEHIDQLWPVLTREPQDHIPWSSLLALPQSYIVPGGRF |
| | | SETYYWDSYFTMLGLAESGREDLLKCMADNFAWMIENYGHIPNGNRTYYLSRSQ |
| | | PPVFALMVELFEEDGVRGARRYLDHLKMEYAFWMDGAESLIPNQAYRHVVRMP |
| | | DGSLLNRYWDDRDTPRDESWLEDVETAKHSGRPPNEVYRDLRAGAASGWDYSS |
| | | RWLRDTGRLASIRTTQFIPIDLNAFLFKLESAIANISALKGEKETEALFRQKASARRD |
| | | AVNRYLWDDENGIYRDYDWRREQLALFSAAAIVPLYVGMANHEQADRLANAVR |
| | | SRLLTPGGILASEYETGEQWDKPNGWAPLQWMAIQGFKMYGDDLLGDEIARSW |
| | | LKTVNQFYLEQHKLIEKYHIADGVPREGGGGEYPLQDGFGWTNGVVRRLIGLYGEP |
| 7 | Cca | MKIYLVGGAVRDALLGLPVKDRDWVVVGSTPQEMLDAGYQQVGRDFPVFLHPQ |
| | | THEEYALARTERKSGSGYTGFTCYAAPDVTLEDDLKRRDLTINALAQDDNGEIIDPY |
| | | NGLGDLQNRLLRHVSPAFGEDPLRVLRVARFAARYAHLGFRIADETLALMREMTH |
| | | AGELEHLTPERVWKETESALTTRNPQVFFQVLRDCGALRVLFPEIDALFGVPAPAK |
| | | WHPEIDTGIHTLMTLSMAAMLSPQVDVRFATLCHDLGKGLTPPELWPRHHGHG |
| | | PAGVKLVEQLCQRLRVPNEIRDLARLVAEFHDLIHTFPMLNPKTIVKLFDSIDAWR |
| | | KPQRVEQLALTSEADVRGRTGFESADYPQGRWLREAWEVAQSVPTKAVVEAGF |
| | | KGVEIREELTRRRIAAVASWKEQRCPKPE |
| 8 | BacA | MSDMHSLLIAAILGVVEGLTEFLPVSSTGHMIIVGHLLGFEGDTAKTFEVVIQLGSIL |
| | | AVVVMFWRRLFGLIGIHFGRPLQHEGESKGRLTLIHILLGMIPAVVLGLLFHDTIKSL |
| | | FNPINVMYALVVGGLLLIAAECLKPKEPRAPGLDDMTYRQAFMIGCFQCLALWPG |
| | | FSRSGATISGGMLMGVSRYAASEFSFLLAVPMMMGATALDLYKSWGFLTSGDIP |
| | | MFAVGFITAFVVALIAIKTFLQLIKRISFIPFAIYRFIVAAAVYVVFF |

A “microbe”, as described herein, can include fungi, bacteria, algae, protozoan, and nematodes. A microbe can be adapted to a specific environment (e.g., a high salinity environment). A microbe can also include any of the nucleic acid constructs described herein for sensing an analyte. For example, an adapted biosensing microbe can be selected from a fungi, a bacterium, an algae, a protozoan, and a nematode. In some cases, the adapted biosensing microbe can be selected from a fungi and a bacterium. In some cases, the adapted biosensing microbe is a fungi. For example, the fungi can be selected of a genera selected from Saccharomyces and Pichia. In some cases, the fungi is of a species selected from Saccharomyces cerevisiae and Pichiapastoris. In some cases, the adapted biosensing microbe is a bacterium. For example, the bacterium is of a genera selected from Escherichia, Streptococcus, Staphylococcus, Salmonella, Campylobacter, Pseudomonas, Bacillus, Klebsiella, and Vibrio. In some cases, the bacterium is from a species selected from Escherichia coli and Staphylococcus aureus.

An adapted biosensing microbe can include any of the nucleic acid constructs described herein. The adapted biosensing microbes can include the nucleic acid constructs on a vector or homologously recombined with the native genome. In some cases, an adapted biosensing microbe includes any of the nucleic acid constructs described herein on a vector. In some cases, an adapted biosensing microbe includes any of the nucleic acid constructs described herein homologously recombined with the native genome.

### Nucleic Acid Constructs

Described herein are nucleic acid constructs that include an inducible promoter operably linked to a gene encoding a signal; a selectable marker; and an origin of replication. In some cases, the inducible promoter is induced by a threshold level of a heavy metal.

A “nucleic acid construct” can include at least one nucleic acid.

As used herein, “nucleic acid” is used to include any compound and/or analyte that comprise a polymer of nucleotides and can include a deoxyribonucleic acid (DNA) or ribonucleic acid (RNA), or a combination thereof, in either a single- or double-stranded form. Nucleotides can have moieties that contain the known purine and pyrimidine bases. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G). Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some cases, nucleic acid constructs include introduced genetic material.

As used herein, “introduced genetic material” means genetic material that is added to, and remains as a component of, the genome of the recipient, including remaining a component of a vector. Introduced genetic material can include exogenous genetic material and recombinant genetic material.

As used herein, the term “exogenous” refers to any material introduced from or originating from outside a microbial cell that is not produced by or does not originate from the same microbial cell in which it is being introduced. As used herein, the term “recombinant” refers to any material introduced from or originating from a different type of nucleic acid. For example, an exogenous nucleic acid is a recombinant nucleic acid. In addition, a recombinant nucleic acid can include a genetic construct using a promoter in a non-native context, i.e., the recombinant nucleic acid includes a promoter that is also found in another location in the genome of the organism and the other location is the promoter's native context.

In some cases, any of the components (e.g., gene, promoter, terminator, origin of replication, etc.) of a nucleic acid construct described herein can be replaced by homologous components. As used herein, “homologous” describes two nucleic acid components with the same function. Homologous genes often have a high sequence identity, for example, 80% sequence identity (e.g., 85%, 90%, 91%, 92%, 93%, 94%, 95%, 96%, 97%, 98%, or 99% sequence identity). Sequence identity, such as for the purpose of assessing percent sequence identity, may be measured by any suitable alignment algorithm, including, but not limited to, the Needleman-Wunsch algorithm (see e.g. the EMBOSS Needle aligner available at ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment may be assessed using any suitable parameters of a chosen algorithm, including default parameters.

As used herein, an “inducible promoter” is a promoter that is under control of an analyte found in the environment (e.g., found in a sample). Examples of environmental conditions that may affect transcription by inducible promoters include certain analytes (e.g., heavy metals), acidic or basic conditions, etc. In some cases, inducible promoters are induced by a heavy metal. Non-limiting examples of inducible promoters include the arsR promoter (P_{arsR}), the cadC promoter (P_{cadC}), the cusC promoter (P_{cusC}), the zntA promoter (P_{zntA}), the mer promoter (Pₘₑᵣ), and the zarP promoter (P_{zarP}). In some cases, the arsR promoter is derived from E. coli, the cadC promoter is derived from Staphylococcus aureus, the cusC promoter is derived from E. coli, the zntA promoter is derived from E. coli, the mer promoter is derived from transposon TN21, or the zarP promoter is from E. coli. In some cases, the arsR promoter is derived from E. coli R773, the cadC promoter is derived from Staphylococcus aureus p1258, the cusC promoter is derived from E. coli MG1655, the zntA promoter is derived from E. coli MG1655, and the zarP promoter is from E. coli MG1655. In some cases, the mer promoter is derived from transposon TN21 is bidirectional. In some cases, the arsR promoter is induced by arsenic, the cadC promoter is induced by cadmium, the cusC promoter is induced by copper, the zntA promoter is induced by lead, the mer promoter is induced by mercury, or the zarP promoter is induced by zinc. See Table 2 for exemplary sequences of heavy-metal inducible promoters. In some cases, inducible promoters have at least 80% sequence identity (e.g., 85%, 90%, 95%, 96%, 97%, 98%, 99% sequence identity) to a sequence described in Table 2.

TABLE 2
Exemplary Heavy-Metal Inducible Promoters.

| SEQ | | |
|---|---|---|
| ID | | |
| NO: | Promoter | Promoter Sequence (5′ to 3′) |
| 9 | P_{arsR} | GTAATAGTGTGATTAATCATATGCGTTTTTGGTTATGTGTTGTTTGACTTAATAT |
| | | CAGAGCCGAGAGATACTTGTTTTCTACAAAGGAGAGGGAAATGTTGCAACTAA |
| | | CACCACTTCAGTTATTTAAAAACCTGTCCGATGAAACCCGTTTGGGTATCGTGTT |
| | | GTTGCTCAGGGAGATGGGAGAGTTGTGCGTGTGTGATCTTTGCATGGCACTGG |
| | | ATCAATCACAGCCCAAAATATCCCGTCATCTGGCGATGCTACGGGAAAGTGGA |
| | | ATCCTTCTGGATCGTAAACAGGGAAAATGGGTTCACTACCGCTTATCACCGCAT |
| | | ATTCCTTCATGGGCTGCCCAGATTATTGAGCAGGCCTGGTTAAGCCAACAGGAC |
| | | GACGTTCAGGTCATCGCACGCAAGCTGGCTTCAGTTAACTGCTCCGGTAGCAGT |
| | | AAGGCTGTCTGCATCTAA |
| 10 | P_{cadC} | CTTACTTCTTTTATTTTCATTCAAATATTTGCTTGCATGATGAGTCGAAAATGGTT |
| | | ATAATACACTCAAATAAATATTTGAATGAAGATGGGATGATAATATGAAAAAA |
| | | AAAGATACCTGCGAAATTTTTTGCTACGATGAAGAAAAAGTGAACCGTATTCAG |
| | | GGAGACTTGCAGACCGTGGATATCTCCGGTGTATCCCAGATTCTGAAGGCGAT |
| | | CGCGGATGAAAATCGTGCCAAGATCACTTATGCGCTGTGCCAGGATGAAGAAC |
| | | TTTGTGTTTGTGATATTGCGAATATTCTCGGGGTGACTATCGCGAATGCATCAC |
| | | ACCACCTGCGCACATTGTACAAACAAGGTGTGGTTAACTTTCGCAAAGAAGGT |
| | | AAATTGGCTCTTTATTCGCTTGGAGACGAACACATCCGCCAAATTATGATGATC |
| | | GCGCTGGCCCATAAAAAAGAAGTAAAAGTGAACGTCTAA |
| 11 | P_{cusC} | TCGCTTATTGGCAAAATGACAATTTTGTCATTTTTCTGTCACCGGAAAATCAGAG |
| | | CCTGGCGAGTAAAGTTGGCGGC |
| 12 | P_{zntA} | GCTGTTTATCAGTAACTTTGTCTGGCTGGGGAGCCACTATCGCCGACGCTTCCG |
| | | TGCGGATAACGCGATTGCTGCGGCCTGCTACTTTGCCGGTCACTTCCTGATCGT |
| | | CCGCTCGCTGTATCTCTGATAAAACTTGACTCTGGAGTCGACTCCAGAGTGTAT |
| | | CCTTCGGTTA |
| 13 | Pₘₑᵣ | TTACGGCATGGCACTACGCGCCAAGCCCGCCTCACCTTGCAGTGACGCAATCAG |
| | | AGGGCAAGACACGTTTCCCTTCCGAGCGTGACAAGCGCACACCAATTCAGAGA |
| | | GCACCGTCTCCATACGCGCCAGGTCCGCCATTTTCTCACGGACATCTTTCAGTTT |
| | | GTGTTCCGCGAGGCTTGACGCCTCTTCACAATGGGTGCCGTCATCCAAACGCAG |
| | | TAACTCTGCAATTTCATCCAGCGAAAAGCCCAGCCGTTGCGCGCTTTTCACGAA |
| | | TTTAACACGCACGACGTCGGCTTCGCCATAACGCCGAATCGAGCCGTATGGCTT |
| | | ATCAGGCTCCCGCAGCAAACCTTTCCGCTGGTAAAAACGGATCGTTTCTACGTT |
| | | CACCCCAGCTGCTTTGGCAAACACCCCAATCGTCAGGTTTTCCAGGTTGTTCTCC |
| | | ATatcgcttgactccgtacatgagtacggaagtaaggttacgctatccaatccaaa |
| | | ttcaaa |
| 14 | P_{zraP} | AATCATGCCATCTTTTATCAGCGCTTACCCTGCGCTGTAACACAAAGGCTTAAGT |
| | | TTCAATGAGTAAAAATGACTCGCTACCCGCAGCAGGCGAGTCATTTTTACTCGT |
| | | TTATCATGCCAGATTACCCGTCATATCAGCGTTTCATCGTTGGCACGGAAGATG |
| | | CAATACCCGAAGTA |

Inducible promoters can be operably linked to a gene encoding a signal (e.g., a biosensing microbe signal). In some cases, heavy-metal inducible promoters are operably linked to a gene encoding a signal that helps determine the amount of the analyte. A signal can be any response that can be quantified using any of the microbe detecting sensors described herein. Non-limiting examples of signal can include a fluorescent signal, a luminescent signal, and a colorimetric signal.

In some cases, the signal is a fluorescent signal. Fluorescent signals can be encoded by a gene encoding any appropriate fluorescent protein. See, for example, any of the fluorescent proteins, or variants thereof, included in the fluorescent protein database named FPbase (fpbase.org). Non-limiting examples of fluorescent proteins include green fluorescent protein (GFP), red fluorescent protein (RFP), cyan fluorescent protein (CFP), yellow florescent protein (YFP), and the like. As shows in Example 2, heavy-metal inducible promoters were operably linked to a green fluorescent protein that provided data to determine the concentration of the heavy metal in a sample. In some cases, genes encoding fluorescent proteins have at least 80% sequence identity (e.g., 85%, 90%, 95%, 96%, 97%, 98%, 99% sequence identity) to a sequence described in Table 3. See Table 3 for exemplary sequences of genes encoding proteins that provide a signal (can be referred to as signal proteins).

As used herein, the term “encode” as it is applied to nucleic acid sequences refers to a nucleic acid sequence which is said to “encode” a polypeptide if, in its native state or when manipulated by methods well known to those skilled in the art, can be transcribed and/or translated to produce the mRNA for the polypeptide and/or a fragment thereof.

When light of a specific wavelength is shown on the fluorescent protein (excitation wavelength), the fluorescent protein produces light at a specific wavelength (emission wavelength). Different fluorescent proteins have different excitation wavelengths and emission wavelengths that are often measured in nanometers (nm). See Table 3 for excitation wavelengths and emission wavelengths of various exemplary fluorescent proteins.

In some cases, the signal is a luminescent signal. Luminescent signals can be encoded by a gene encoding any appropriate luciferase protein. Luciferase is a generic term for the class of oxidative enzymes that produce luminescence. A variety of luciferase proteins are known in the art. (See, for example, Fleiss and Sarkisyan. Curr. Genetics. 65, pages 877-882 (2019)).

In some cases, the signal is a colorimetric signal. Colorimetric signals can be encoded by a gene encoding any protein that produces a colored produce during a chemical reaction.

TABLE 3
Exemplary Signal Proteins.

| SEQ | | Excitation | Emission | |
|---|---|---|---|---|
| ID | Signal | Wavelength | Wavelength | Amino Acid Sequence |
| NO: | Protein | (nm) | (nm) | (5′ to 3′) of Signal Protein |
| 15 | Super Fold | 485 | 510 | MSKGEELFTGVVPILVELDGDVNGHKFSVRG |
| | Green | | | EGEGDATNGKLTLKFICTTGKLPVPWPTLVTT |
| | Fluorescent | | | LTYGVQCFSRYPDHMKRHDFFKSAMPEGYVQ |
| | Protein | | | ERTISFKDDGTYKTRAEVKFEGDTLVNRIELK |
| | (sfGFP) | | | GIDFKEDGNILGHKLEYNFNSHNVYITADKQK |
| | | | | NGIKANFKIRHNVEDGSVQLADHYQQNTPIG |
| | | | | DGPVLLPDNHYLSTQSVLSKDPNEKRDHMVL |
| | | | | LEFVTAAGITHGMDELYK |
| 16 | Red | 588 | 633 | MSELIKENMHMKLYMEGTVNNHHFKCTSEG |
| | Fluorescent | | | EGKPYEGTQTMRIKVVEGGPLPFAFDILATSF |
| | Protein | | | MYGSKTFINHTQGIPDFFKQSFPEGFTWERVT |
| | (RFP) | | | TYEDGGVLTATQDTSLODGCLIYNVKIRGVNF |
| | | | | PSNGPVMQKKTLGWEASTEMLYPADGGLEG |
| | | | | RSDMALKLVGGGHLICNLKTTYRSKKPAKNLK |
| | | | | MPGVYYVDRRLERIKEADKETYVEQHEVAVAR |
| | | | | YCDLPSKLGHK |
| 17 | Cyan | 456 | 480 | MSKGEELFTGVVPILVELDGDVNGHKFSVSGE |
| | Fluorescent | | | GEGDATYGKLTLKFICTTGKLPVPWPTLVTTFS |
| | Protein | | | WGVQCFSRYPDHMKQHDFFKSAMPEGYVQER |
| | (CFP) | | | TIFFKDDGNYKTRAEVKFEGDTLVNRIELKGID |
| | | | | FKEDGNILGHKLEYNYNSHNVYIMADKQKNGI |
| | | | | KVNFKIRHNIEDGSVQLADHYQQNTPIGDGPV |
| | | | | LLPDNHYLSTQSALSKDPNEKRDHMVLLEFVT |
| | | | | AAGITHGMDELYK |
| 18 | Yellow | 514 | 529 | MVSKGEELFTGVVPILVELDGDVNGHKFSVRG |
| | Fluorescent | | | EGEGDATNGKLTLKLISTTGKLPVPWPTLVTTL |
| | Protein | | | GYGLMVFARYPDHMKQHDFFKSAMPEGYVQE |
| | (YFP) | | | RTISFEDDGYYKTRAEVKFEGDTLVNRIVLKGI |
| | | | | DFKEDGNILGHKLEYNFNSHNVYITADKQKNG |
| | | | | IKANFKIRHNVEDGGVQLADHYQQNTPIGDG |
| | | | | PVLLPDNHYLSYQSVLSKDPNEKRDHMVLKER |
| | | | | VTAAGITHDMNELYK |

As used herein, the term “operably linked” refers to the association of a nucleic acid sequence with a second nucleic acid sequence containing a promoter on a single nucleic acid fragment so that the function of the nucleic acid sequence is regulated by the second nucleic acid sequence containing the promoter. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

Any of the nucleic acid constructs described herein can also include additional regulation components, such as genes encoding a transcriptional activator, to modulate expression of the signal. Transcriptional activators are proteins that bind to DNA and stimulate transcription of nearby genes. Many activators enhance RNA polymerase binding (formation of the closed complex) or the transition to the open complex required for initiation of transcription. Transcriptional activators can interact directly with a subunit of RNA polymerase. Exemplary genes encoding transcriptional activators that bind to heavy-metal inducible promoters are included in Table 4. In some cases, transcriptional activators have at least 80% sequence identity (at least 90%, 95%, 96%, 97%, 98%, 99% sequence identity) to any of the sequences of Table 4.

TABLE 4
Exemplary genes encoding transcriptional activators.

| SEQ | Transcriptional | |
|---|---|---|
| ID | Activator | |
| NO: | Gene | Nucleic Acid Sequence (5′-3′) |
| 19 | ArsR | ATGTTGCAACTAACACCACTTCAGTTATTTAAAAACCTGTCCGATGAAA |
| | | CCCGTTTGGGTATCGTGTTGTTGCTCAGGGAGATGGGAGAGTTGTGCG |
| | | TGTGTGATCTTTGCATGGCACTGGATCAATCACAGCCCAAAATATCCCG |
| | | TCATCTGGCGATGCTACGGGAAAGTGGAATCCTTCTGGATCGTAAACA |
| | | GGGAAAATGGGTTCACTACCGCTTATCACCGCATATTCCTTCATGGGCT |
| | | GCCCAGATTATTGAGCAGGCCTGGTTAAGCCAACAGGACGACGTTCAG |
| | | GTCATCGCACGCAAGCTGGCTTCAGTTAACTGCTCCGGTAGCAGTAAG |
| | | GCTGTCTGCATCTAA |
| 20 | CadC | ATGAAAAAAAAAGATACCTGCGAAATTTTTTGCTACGATGAAGAAAAA |
| | | GTGAACCGTATTCAGGGAGACTTGCAGACCGTGGATATCTCCGGTGTA |
| | | TCCCAGATTCTGAAGGCGATCGCGGATGAAAATCGTGCCAAGATCACT |
| | | TATGCGCTGTGCCAGGATGAAGAACTTTGTGTTTGTGATATTGCGAATA |
| | | TTCTCGGGGTGACTATCGCGAATGCATCACACCACCTGCGCACATTGTA |
| | | CAAACAAGGTGTGGTTAACTTTCGCAAAGAAGGTAAATTGGCTCTTTAT |
| | | TCGCTTGGAGACGAACACATCCGCCAAATTATGATGATCGCGCTGGCC |
| | | CATAAAAAAGAAGTAAAAGTGAACGTCTAA |
| 21 | MerR | TTACGGCATGGCACTACGCGCCAAGCCCGCCTCACCTTGCAGTGACGC |
| | | AATCAGAGGGCAAGACACGTTTCCCTTCCGAGCGTGACAAGCGCACAC |
| | | CAATTCAGAGAGCACCGTCTCCATACGCGCCAGGTCCGCCATTTTCTCA |
| | | CGGACATCTTTCAGTTTGTGTTCCGCGAGGCTTGACGCCTCTTCACAAT |
| | | GGGTGCCGTCATCCAAACGCAGTAACTCTGCAATTTCATCCAGCGAAA |
| | | AGCCCAGCCGTTGCGCGCTTTTCACGAATTTAACACGCACGACGTCGG |
| | | CTTCGCCATAACGCCGAATCGAGCCGTATGGCTTATCAGGCTCCCGCA |
| | | GCAAACCTTTCCGCTGGTAAAAACGGATCGTTTCTACGTTCACCCCAGC |
| | | TGCTTTGGCAAACACCCCAATCGTCAGGTTTTCCAGGTTGTTCTCCAT |

Any of the nucleic acid constructs described herein can also include a selectable marker. Such selectable markers allow researchers to assess successful incorporation of the nucleic acid constructs on which the selectable marker was included. Any appropriate selectable marker known in the art can be used in any of the nucleic acid constructs or vectors described herein. For example, selectable markers can include antibiotic selectable markers. Antibiotic selectable markers are encoded by genes that are known in the art, and confer resistance to a specific antibiotic or toxin. An antibiotic resistant gene can be incorporated into the genome of a microbe or a vector to be incorporated into a microbe using any known methods (e.g., transformation of a vector with a selectable antibiotic marker). Non-limiting examples of antibiotic selectable markers include ampicillin, kanamycin, chloramphenicol, erythromycin, spectinomycin, neomycin, streptomycin, zeocin, and gentamicin. In some cases, the antibiotic selectable marker is kanamycin.

Any of the nucleic acid constructs described herein can also include an origin of replication. Origins of replication (also called the replication origin) is a particular nucleic acid sequence in a genome at which replication is initiated. Inclusion of an origin of replication on a nucleic acid construct (e.g., a vector), ensures that the nucleic acid construct can be replicated inside the microbe in which it is found. Any appropriate origin of replication that facilitates replication of a nucleic acid construct in a microbe can be used. In some cases, the origin of replication is p15A. p15A is a well-known origin of replication for bacteria, such as E. coli.

Any of the nucleic acid constructs described herein can also include terminators (also known as transcription terminators). A terminator is a section of a nucleic acid sequence that marks the end of a gene or operon in genomic DNA during transcription. This sequence mediates transcriptional termination by providing signals in the newly synthesized transcript RNA that trigger processes which release the transcript RNA from the transcriptional complex.

Any of the nucleic acid constructs or any of the nucleic acid sequences of the nucleic acid constructs can be codon-optimized for expression in a specific microbial cell. Different organisms prefer certain codons over others for translation into amino acids. This is termed codon usage bias. In fact, some species are known to avoid certain codons altogether. Codon optimization can improve gene expression by changing synonymous codons based on an organism's codon bias. Point mutations can be made throughout a gene of interest based on an organism's codon usage bias to increase translational efficiency and therefore protein expression without altering the translated amino acid sequence. Any codon-optimization algorithms or resources can be used to identify codon-optimized nucleic acid sequences. Examples in include codon-optimization tools offered by IDT (idtdna.com/pages/tools/codon-optimization-tool?returnurl=%2FCodonOpt) and GenScript (genscript.com/tools/rare-codon-analysis).

Genetically engineered microbes described herein can be intergeneric microbes, non-intergeneric microbes, or intrageneric microbes. As used herein, an “intergeneric microbe” is a microbe that is formed by the deliberate combination of genetic material originally isolated from organisms of different taxonomic genera. An exemplary “intergeneric microbe” includes a microbe containing a genetic element which was first identified in a microbe in a genus different from the recipient microbe. Further explanation can be found, inter alia, in 40 C.F.R. § 725.3. In some cases, adapted microbes described herein are “non-intergeneric,” which means that the microbes are not intergeneric. As used herein, an “intrageneric microbe” is a microbe that is formed by the deliberate combination of genetic material originally isolated from organisms of the same taxonomic genera.

As used herein, the term “expression” refers to the process by which polynucleotides are transcribed into mRNA and/or the process by which the transcribed mRNA is subsequently translated into peptides, polypeptides, or proteins.

### Methods of Producing Adapted Biosensing Microbes

Any of the methods, systems, products, or compositions described herein can include an adapted biosensing microbe to determine an amount of an analyte in a sample. High salinity, as described above, can cause high noise and/or a high baseline when detecting an analyte with a biosensing microbe (e.g., an adapted biosensing microbe). An adapted biosensing microbe can be adapted to, for example, increase sensitivity and/or increase accuracy when detecting an analyte in a sample of a specific medium type (e.g., a high salinity medium) as compared to a non-adapted microbe (also known as ancestral microbe).

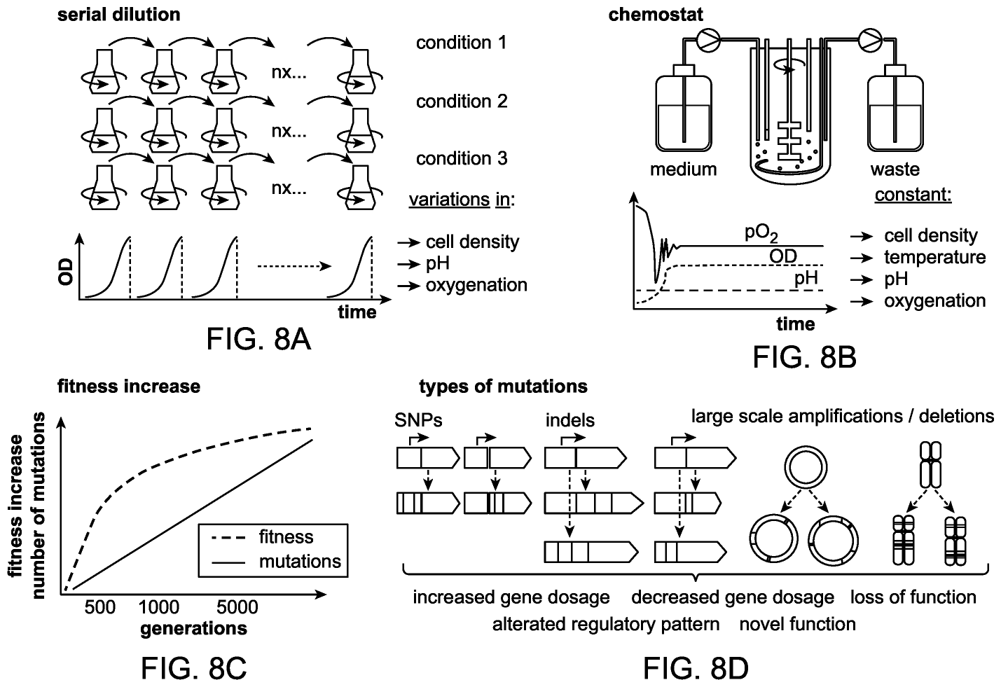

Described herein are methods of producing an adapted microbe, which can also be an adapted biosensing microbe used to detect an analyte in a sample. Adaptation to a selected medium, such as a high salinity medium, can decrease the noise associated with detecting an amount of an analyte, and/or can decrease the baseline associated with detecting an amount of an analyte. Decreasing the noise and/or baseline associated with detecting an amount of an analyte can, for example, increase accuracy and sensitivity of detecting the analyte (e.g., detecting an amount of a heavy metal with an adapted biosensing microbe). Adaptation of a biosensing microbe can use adapted laboratory evolution (ALE). (See, for example, Dragosits and Mattanovich. Microbial Cell Factories. 12:64). ALE can include growth of a microbe and serial dilution in a specified medium type (e.g., a high salinity medium). See FIG. 8 for an exemplary schematic of serial dilutions. Serial dilution can use a series of dilution and subsequent growth in shaking flasks, growth in a chemostat, or growth in a microfluidic device.

In some cases, the method of producing an adapted biosensing microbe to detect an analyte in a sample can include obtaining a non-adapted biosensing microbe; growing the non-adapted biosensing microbe in a medium similar to the sample to generate one or more adapted biosensing microbes; selecting the adapted biosensing microbe from the one or more adapted biosensing microbes by selecting for one or more traits selected from the group of increased growth rate compared to the non-adapted biosensing microbe, increased yield compared to the non-adapted biosensing microbe, increased sensitivity of detection of the analyte compared to the non-adapted biosensing microbe, increased production of the signal compared to the non-adapted biosensing microbe, decreased baseline of the signal in the absence of the analyte compared to the non-adapted biosensing microbe, decreased biofilm formation compared to the non-adapted biosensing microbe, increased synthesis of osmoprotectants compared to the non-adapted biosensing microbe, increased uptake of osmoprotectants compared to the non-adapted biosensing microbe, and increased resistance to toxicity associated with increased levels of osmoprotectants compared to the non-adapted biosensing microbe, thereby producing an adapted biosensing microbe to detect an analyte in a sample.

In some cases, the method of producing an adapted biosensing microbe to detect an analyte in a sample can also include, after selecting the adapted biosensing microbe from the one or more adapted biosensing microbes, serially-passaging the selected adapted biosensing microbe at least once to fresh medium to generate a further adapted biosensing microbe. A further adapted biosensing microbe can have a similar selected trait of the adapted biosensing microbe, an additional selected trait of any of the selected traits, or an enhanced selected trait compared to the adapted biosensing microbe. Serially-passaging the adapted biosensing microbe or further adapted biosensing microbe can generate additional selected traits and/or enhanced selected traits. In some cases, serially-passaging the adapted biosensing microbe or further adapted biosensing microbe can use fresh medium that is the same type of medium in which the non-adapted microbe was grown. In some cases, serially-passaging the adapted biosensing microbe or further adapted biosensing microbe can use fresh medium that is the a different type of medium in which the non-adapted microbe was grown.

Adapted biosensing microbes can include any of the nucleic acid constructs described herein.

### Systems For Use In Determining An Amount of An Analyte in A Sample

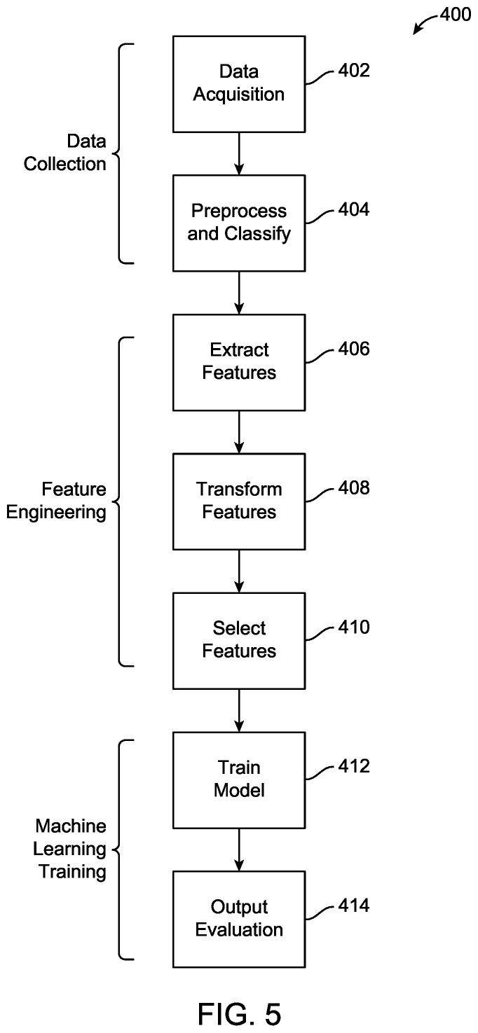

FIG. 5 shows an example process 400 that can be used to produce classifiers that are able to evaluate data received from the adapted biosensing microbe (e.g., the biosensing microbe signal) and determine an amount of an analyte in a sample. For example, the process 400 can be performed by the elements of the system described in 300 and will be described with reference to those elements. However, other systems can be used to perform the process 400 or similar processes.

Generally speaking, the process 400 includes data collection 402-404, feature engineering 406-410, and machine learning training 412-414. In the data collection 402-404, data is gathered in formats in which it is generated or transmitted, then reformatted, decorated, aggregated, or otherwise processed for use. In the feature engineering 406-410, data is analyzed to find those portions of the data that are sufficiently determine the amount of an analyte to be used to train the classifiers. This can allow the discarding of extraneous or unneeded data, improving computational efficiency and/or accuracy. The machine learning training 412-414 can then use those features to build one or more models that characterize relationships in the data for use in future classifications.

In the data acquisition 402 for example, the computing hardware 310 can collect data from the sensors 304. As will be understood, this acquisition may happen over various lengths of time and some data may be collected after other data is collected.

In the preprocessing and classifying 404 for example, the computing hardware 310 can perform operations to change the format or representation of the data. In some cases, this may not change the underlying data (e.g., changing integers to equivalent floating point numbers, marking the induction period in time-series data), may destroy some underlying data (e.g., reducing the length of binary strings used to represent floating point numbers, applying filters to time-series data), and/or may generate new data (e.g., calculating the fold-change of the response, rate of response (first derivative), or response relaxation behavior post-induction).

In the feature extraction 406 for example, the computing hardware 310 can extract features from the processed and classified data. Some of these features can be related to fold-change of the response, rate of response, or response relaxation behavior post-induction. Features can include averages, standard deviations, and other statistical descriptive.

In the feature transformation 408 for example, the computing hardware 312 can modify the features in ways that preserve all data, destroy some data, and/or generate new data.

In the feature selection 410 for example, the computing hardware 312 can select some of the features for use in training the model. This can include selecting a proper subset (e.g., some, but not all) of the features.

In the model training 412 for example, the computing hardware 312 can train one or more machine-learning models using the selected features. In some cases, one or more models are created that propose mappings between the features and data indicating an amount of a heavy metal for those features. Then, the computing hardware 312 modifies those mappings to improve the model's accuracy.

In the output evaluation 414 for example, the computing hardware 312 can generate one or more functions, sometimes called classifiers, which include a model. This inclusion can involve including the whole model, or may involve only including instructions generated from the model allowing for the classifier to have a smaller memory footprint than the model itself. The machine-learning models (also referred to as machine-learning algorithms or amount classifiers) can include supervised machine learning algorithms. Exemplary and non-limiting machine learning techniques include recurring neural networks, long short term memory, time series forest classifier, and shapelet-based classifier. In some cases, the machine learning techniques include at least one of the group selected from recurring neural networks, long short term memory, time series forest classifier, and shapelet-based classifier.

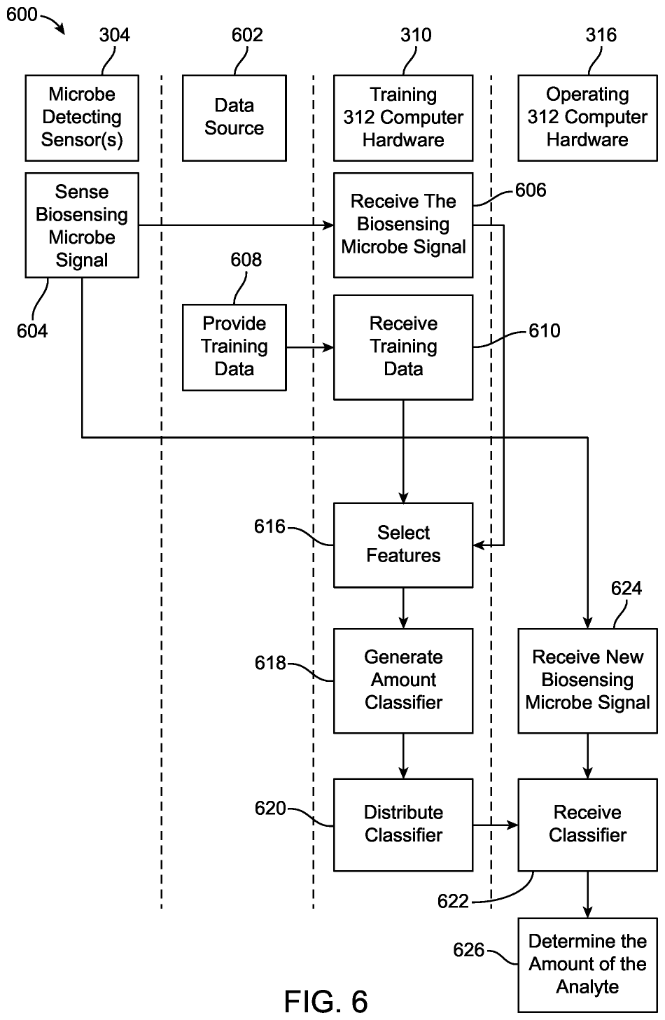

FIG. 6 shows an example process 600 for determining an amount of an analyte. The process 600 can be performed, for example, by the microbe sensors 304, a data source 602, the training computer hardware 310, and the operations computer hardware 316, though other components may be used to perform the process 600 or other similar processes.

The microbe detecting sensor(s) 304 sense the signal (e.g., the biosensing microbe signal (e.g., adapted biosensing microbe signal)) 604 and send the biosensing microbe signal to the training computer hardware 310. The training computer hardware 310 receives the signal of the biosensing microbe (e.g., adapted biosensing microbe) recorded at least partly when inducing the biosensing microbe (e.g., the adapted biosensing microbe). For example, one or more analytes can be sensed using at least one biosensing microbe (e.g., adapted biosensing microbe) to build training data for the process 600. The signal (e.g., biosensing microbe signal) can be analyzed to produce a variety of data, including fold-change of the response, rate of response, or response relaxation behavior post-induction.

The data source 602 provides training data 608 to the training computer hardware 310 and the training computer hardware 310 receives the training data for the subject. For example, the data sources 602 may include a database stored in one or more servers connected to the training computer hardware 310 over a data network such as the internet.

In some cases, the training data includes an amount of an analyte (e.g., a heavy metal), the identity of the biosensing microbe (e.g., the adapted biosensing microbe) that detected the amount of the analyte, genetic constructs and/or nucleic acid constructs of the biosensing microbe, and/or other data associated with the analyte orthe biosensing microbe.

The training computer hardware 310 selects 616 a subset of the biosensing microbe signal or derivatives thereof as selected features. For example, one or more analyses may be performed to identify identifying the subset of the biosensing microbe signal features as those features most predictive of the amount of the analyte to be determined in the sample.

The training computer hardware 310 generates 618 one or more amount classifiers comprising training a model that defines at least one relationship between the biosensing microbe signal and the amount of the analyte to be determined. For example, the model may predict new results based on old training data. The training can include determining hyperparmeters of the model or hyperparameters that control learning processes for a model using a Bayesian optimization algorithm. This optimization algorithm can be configured to target various targets or loss functions, such as a model's performance in repeated k-fold cross-validation. The training can use, for example, a regression; a regression with a loss function based on a residual-label covariance analysis, or a deep label distribution algorithm.

The training computer hardware 310 distributes 620 amount classifiers to a plurality of user devices (e.g., operating computer hardware 316) that are receive the amount classifier 622 and are configured to sense new biosensing microbe signals of other samples in which an amount of an analyte is to be determined. For example, with this amount classifier created, a manufacturer of a device such as a microfluidic device or plate reader device can include the amount classifier in the computing hardware of the device or an application associated with the device to run on a phone, computer, or other device.

The operating computer hardware 316 provides 626, as output, an amount of the analyte determined by the defined relationship between the biosensing microbe signal and the amount of an analyte to be determined. For example, the user may be provided with a report showing the amount of the analyte detected (e.g., the heavy metal detected) on a computer screen, via a mobile application, or in a printed report.

To create this metric for the user, the hardware 316 can submit the new biosensing microbe signal (e.g., new adapted biosensing microbe signal) to at least one of the amount classifiers as the input; and receive as output from the at least one amount classifier the amount of the analyte detected (e.g., the heavy metal detected). In addition to a single metric, the amount classifier can also provide other types of output including but not limited to a confidence value, a model interpretation, a human-readable instruction displayable to a user of an output device, and an automation-instruction that, when executed by an automated device causes the automated device to actuate.

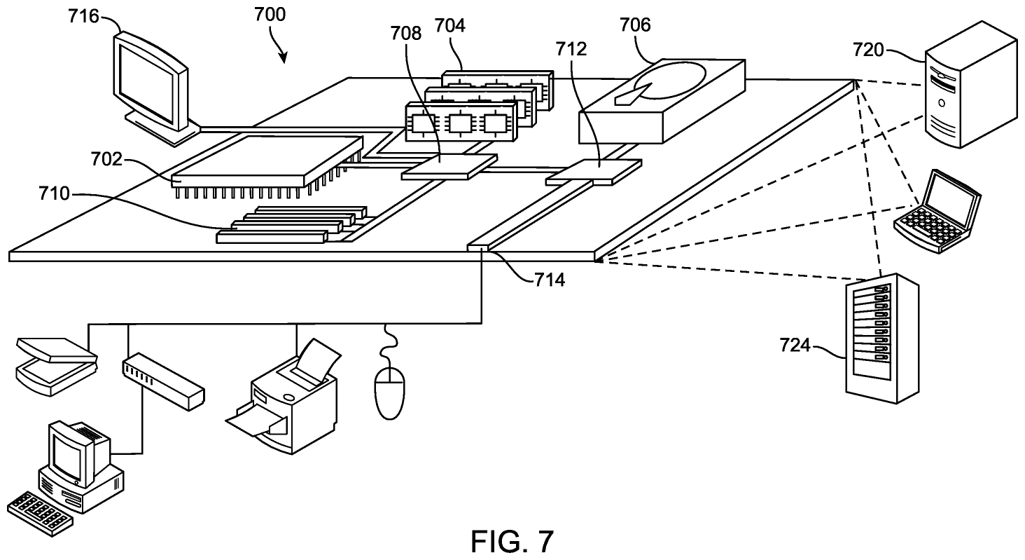

FIG. 7 shows an example of a computing device 700 that can be used to implement the techniques described herein. The computing device 700 is intended to represent various forms of digital computers, such as laptops, desktops, workstations, personal digital assistants, servers, blade servers, mainframes, microbe detecting sensors, and other appropriate computers. The components shown here, their connections and relationships, and their functions, are meant to be exemplary only, and are not meant to limit implementations described and/or claimed in this document.

The computing device 700 includes a processor 702, a memory 704, a storage device 706, a high-speed interface 708 connecting to the memory 704 and multiple high-speed expansion ports 710, and a low-speed interface 712 connecting to a low-speed expansion port 714 and the storage device 706. Each of the processor 702, the memory 704, the storage device 706, the high-speed interface 708, the high-speed expansion ports 710, and the low-speed interface 712, are interconnected using various busses, and can be mounted on a common motherboard or in other manners as appropriate. The processor 702 can process instructions for execution within the computing device 700, including instructions stored in the memory 704 or on the storage device 706 to display graphical information for a GUI on an external input/output device, such as a display 716 coupled to the high-speed interface 708. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 704 stores information within the computing device 700. In some implementations, the memory 704 is a volatile memory unit or units. In some implementations, the memory 704 is a non-volatile memory unit or units. The memory 704 can also be another form of computer-readable medium, such as a magnetic or optical disk.

The storage device 706 is capable of providing mass storage for the computing device 700. In some implementations, the storage device 706 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 704, the storage device 706, or memory on the processor 702.

The high-speed interface 708 manages bandwidth-intensive operations for the computing device 700, while the low-speed interface 712 manages lower bandwidth-intensive operations. Such allocation of functions is exemplary only. In some implementations, the high-speed interface 708 is coupled to the memory 704, the display 716 (e.g., through a graphics processor or accelerator), and to the high-speed expansion ports 710, which can accept various expansion cards (not shown). In the implementation, the low-speed interface 712 is coupled to the storage device 706 and the low-speed expansion port 714. The low-speed expansion port 714, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 700 can be implemented in a number of different forms, as shown in the figure. For example, it can be implemented as a standard server 720, or multiple times in a group of such servers. In addition, it can be implemented in a personal computer such as a laptop computer 722. It can also be implemented as part of a rack server system 724. Alternatively, components from the computing device 700 can be combined with other components in a mobile device (not shown). Each of such devices can contain one or more of the computing device 700, and an entire system can be made up of multiple computing devices communicating with each other.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above.

It must be noted that, as used in the specification and the appended claims, the singular forms “a,” “an” and “the” include plural referents unless the context clearly dictates otherwise.

As used herein, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Various non-limiting aspects of these methods, systems, and compositions are described herein, and can be used in any combination without limitation. Additional aspects of various components of methods, systems, and compositions for detecting an analyte in a sample are known in the art.

### EXAMPLES

### Example 1a—Generating Adapted Biosensing E. coli Strains

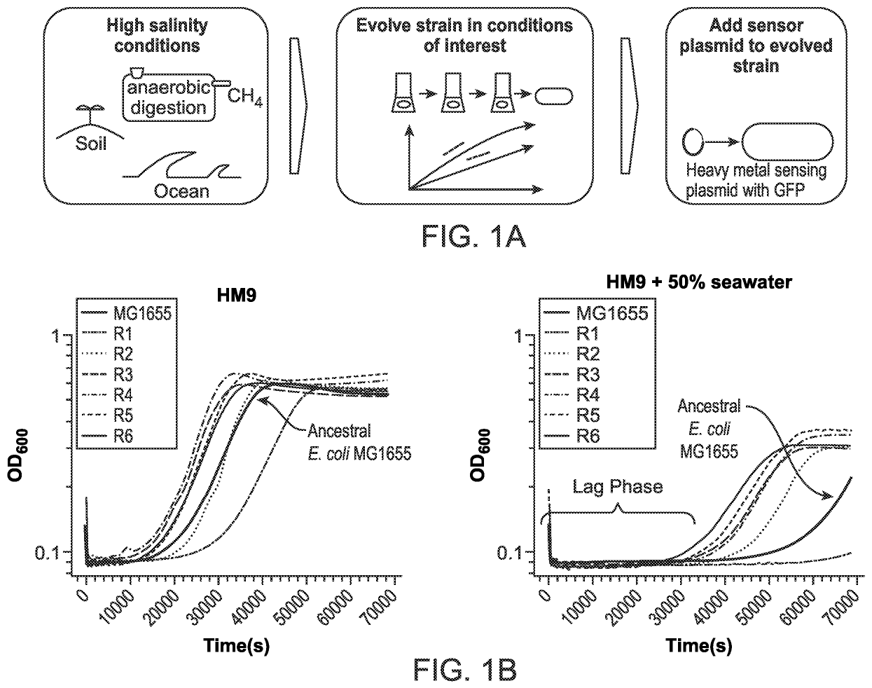

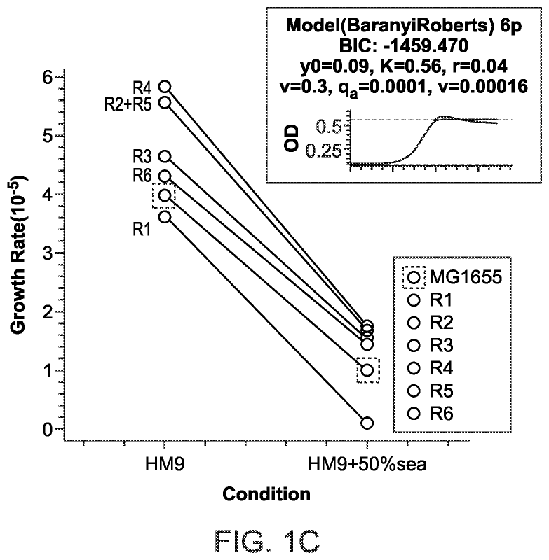

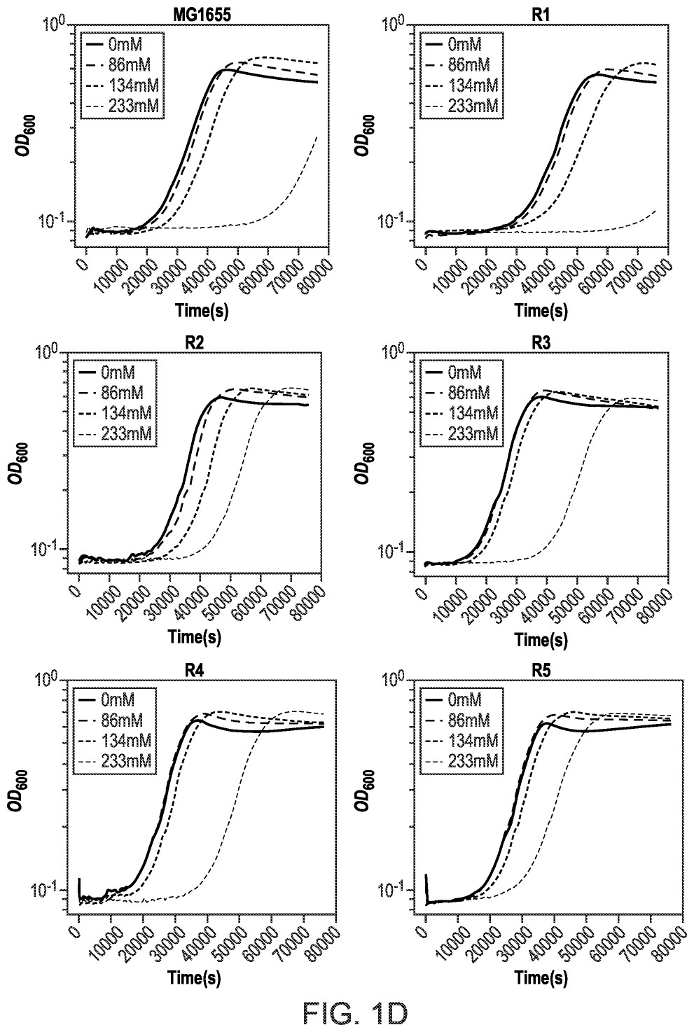

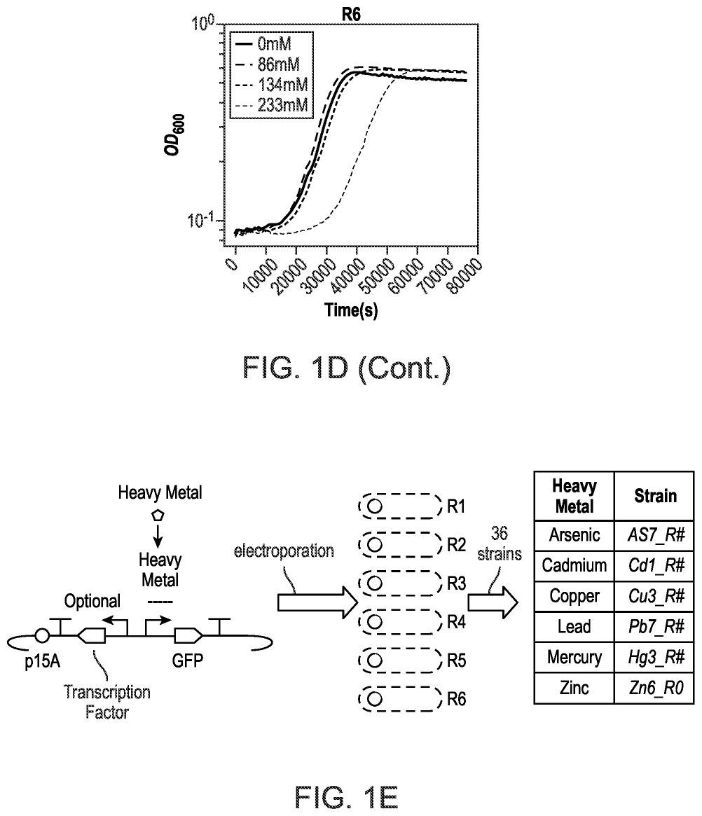

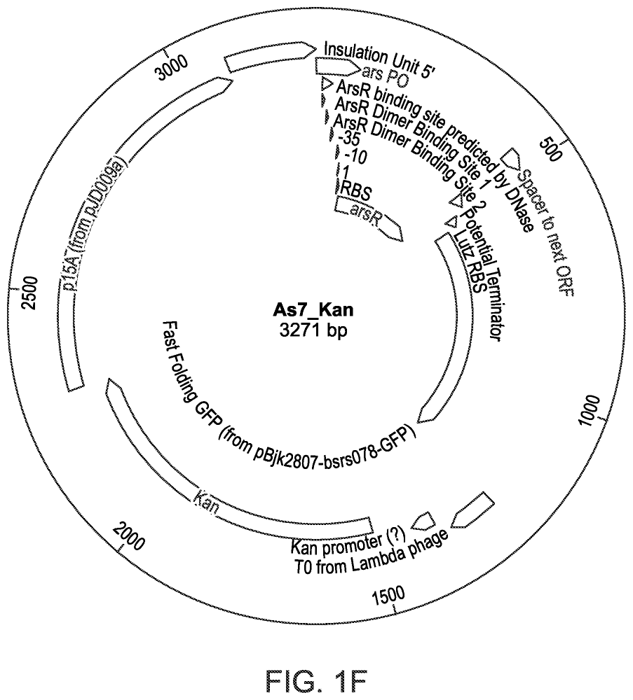

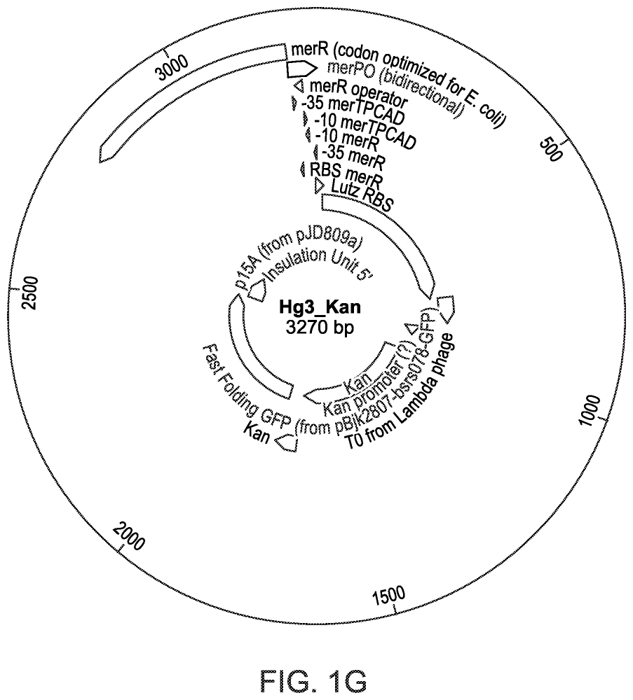

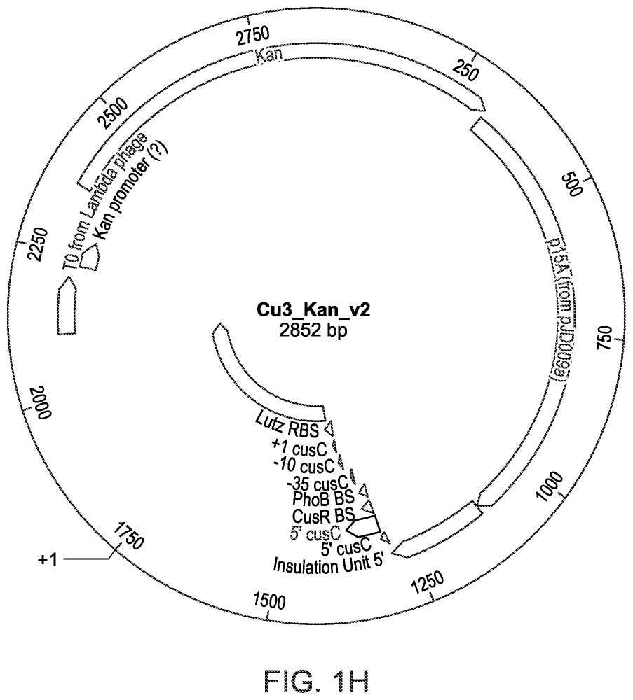

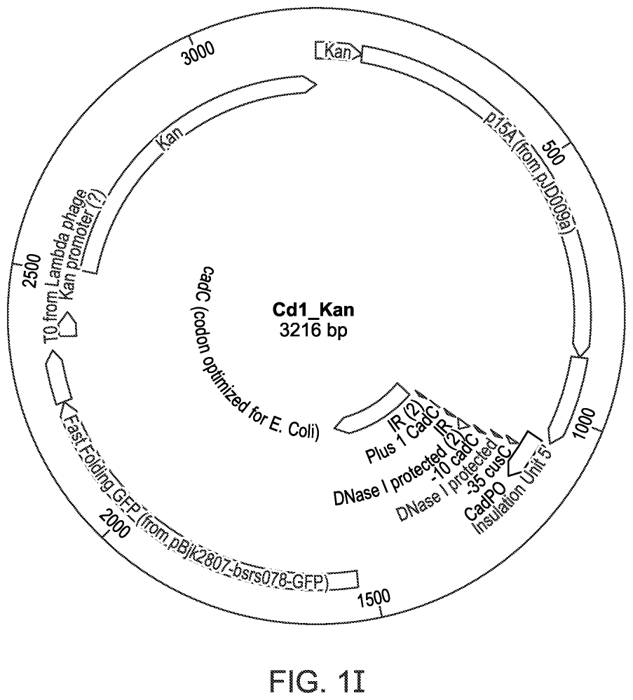

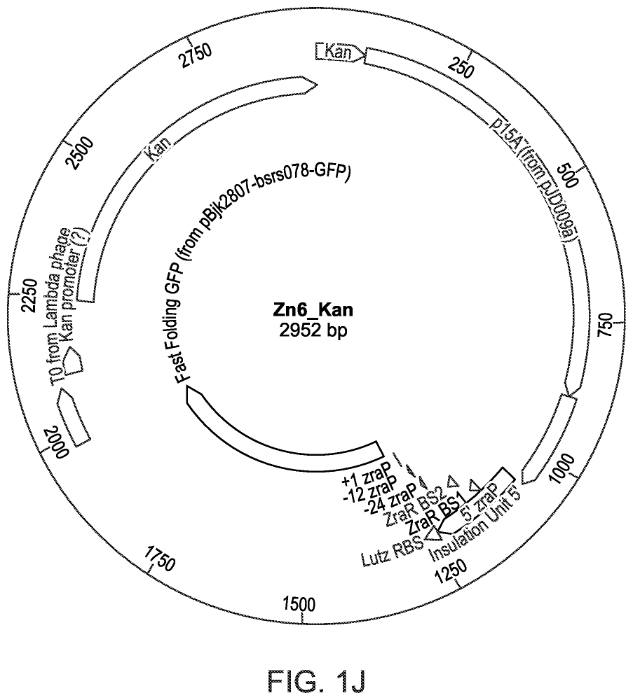

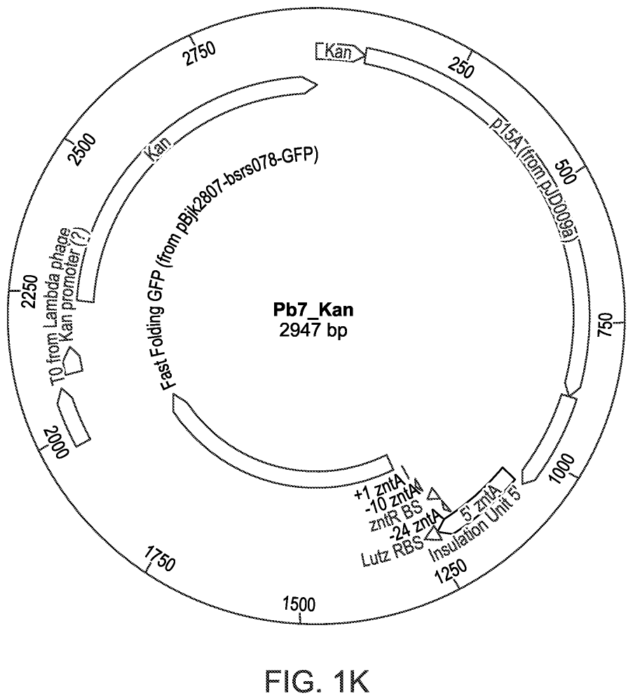

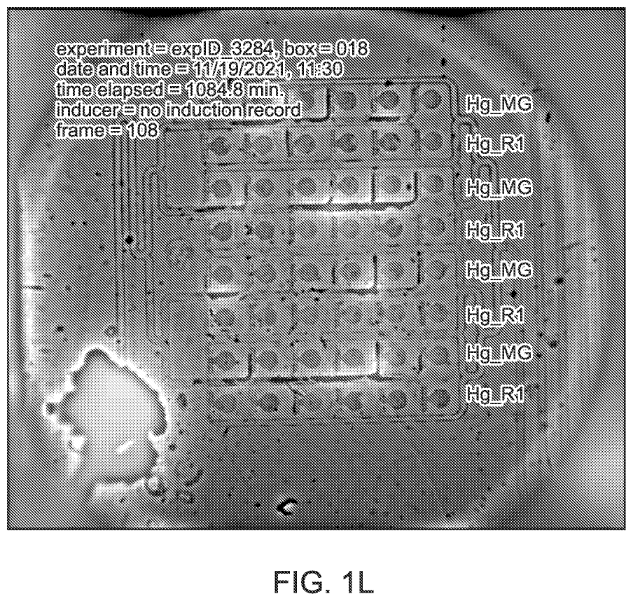

This example describes generating biosensing microbes using adaptive laboratory evolution (ALE) combined with synthetic biology and genetic engineering to develop bacterial biosensing strains that can withstand a high salinity environment (FIG. 1A). Escherichia coli strain MG1655 (ancestral strain) was subjected to a controlled batch culture ALE process using successive passages in minimal media with 50% seawater to evolve six adapted E. coli strains (R1-R6). (See, for example, Sandberg et al. Metabolic Engineering. 56:pages 1-16 (2019)). The adapted E. coli strains were characterized and compared to the ancestral E. coli strain MG1655. Generally, the adapted E. coli strains grew better than the ancestral strain in high salinity environments. Specifically, adapted strains 1) showed a shorter lag phase (time to exponential growth) than the ancestral E. coli strain MG1655 (FIG. 1B), 2) R2-R6 had a higher growth rate than the ancestral E. coli strain MG1655 (FIG. 1C), 3) formed less sticky biofilms, which allowed microfluidic experiments to run without clogging (FIG. 1L), and 4) were more robust in increased salt concentration (FIG. 1D). Growth rates were computed with Baranyi-Roberts growth model, a logistic growth model accounting for adjustment period before logistic growth. (See, for example, Baranyi and Roberts. Int. J. Food Microbiol., 23(3-4):277-94 (1994).)

An analysis of mutations that accumulated during adaptation to HM9 minimal media+50% sea water identified a series of mutations that may have contributed sea water adaptation. Specifically of interest are the genes nagA, ompC, cueO, and phoE, kup, treF, cca, and bacA.

Then, each of R1-R6 adapted E. coli strain was transformed via electroporation independently with six heavy metal sensing plasmids engineered to produce green fluorescent protein (super folder GFP) in the presence of arsenic, cadmium, copper, lead, mercury, or zinc, to yield a total of 36 adapted biosensing E. coli strains (FIG. 1E). Some plasmids contained transcription factors. See Table 5 and FIG. 1F for additional plasmid and transformed strain information.

TABLE 5
Adapted biosensing E. coli strains.

| Heavy | Strain | | |
|---|---|---|---|
| Metal | Name^{A} | Promoter | Transcription Factor |
| Arsenic | As7_R# | P_{arsR} from E. coli R773 | ArsR from E. coli R773 |
| Cadmium | Cd1_R# | P_{cadc} from S. aureus pl258 | CadC from S. aureus pl258 |
| | | | (codon optimized) |
| Copper | Cu3_R# | P_{cusc} from E. coli MG1655 | None |
| Lead | Pb7_R# | P_{zntA} from E. coli MG1655 | None |
| Mercury | Hg3_R# | Pₘₑᵣ from transposon Tn21 | MerR from Tn21 (codon |
| | | (bidirectional) | optimized) |
| Zinc | Zn6_R# | P_{zraP} from E. coli MG1655 | None |

^{A}R# can be R1-R6, referring to the adapted E. coli strain that was transformed with the heavy metal sensing plasmid.

### Example 1b—Modifications of Interest in Adapted Biosensing E. coli Strains

After adapting a microbe as described in Example 1, whole genome sequencing can be performed. In some cases, there is a modification, either a point mutation or frameshift, in the gene of interest, which leads to loss of function, altered in function, gain of function, or altered interactions between proteins and their target (e.g, proteins, RNA, DNA) within the cell. Modifications are found in the genes nagA, ompC, cueO, and phoE, kup, treF, cca, and bacA.

### Example 2—Characterizing Responses of Adapted Biosensing E. coli Strains to Heavy Metals

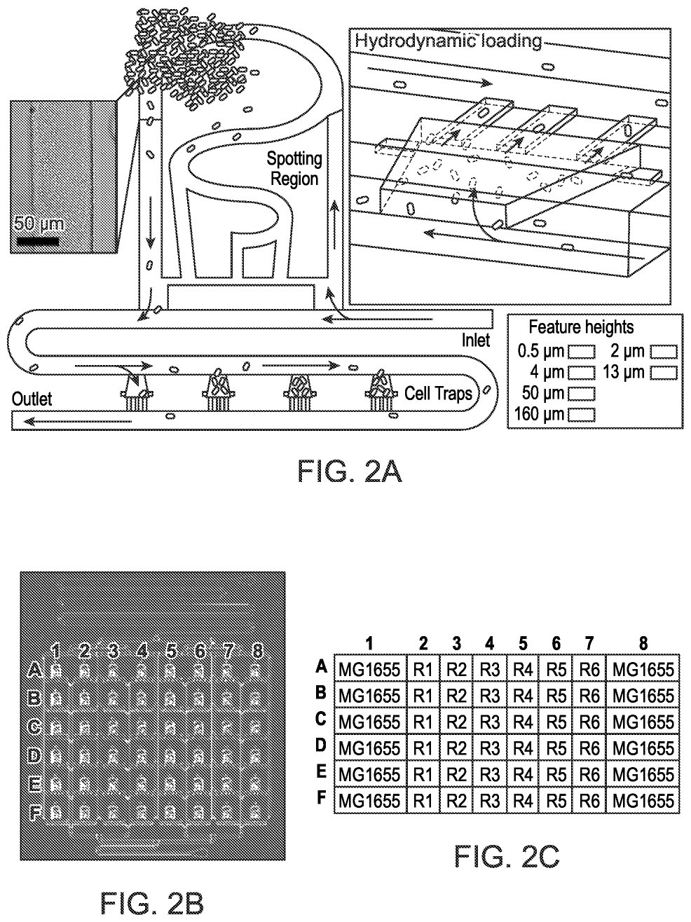

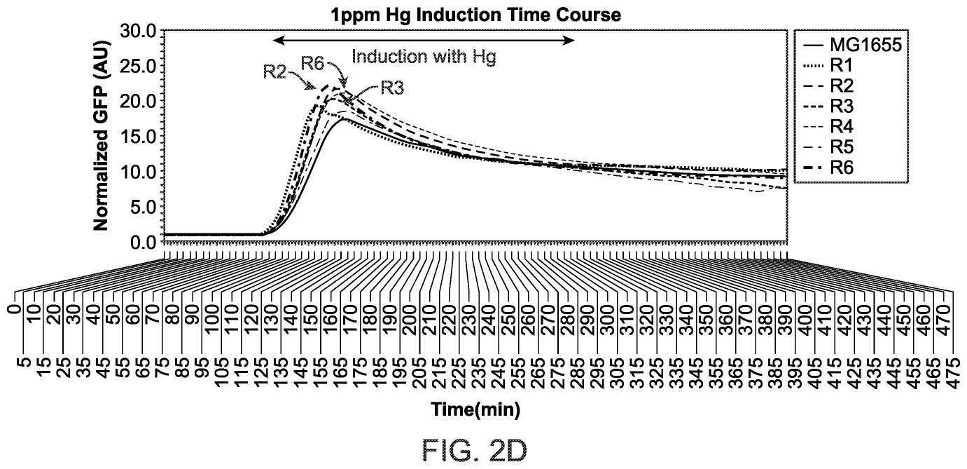

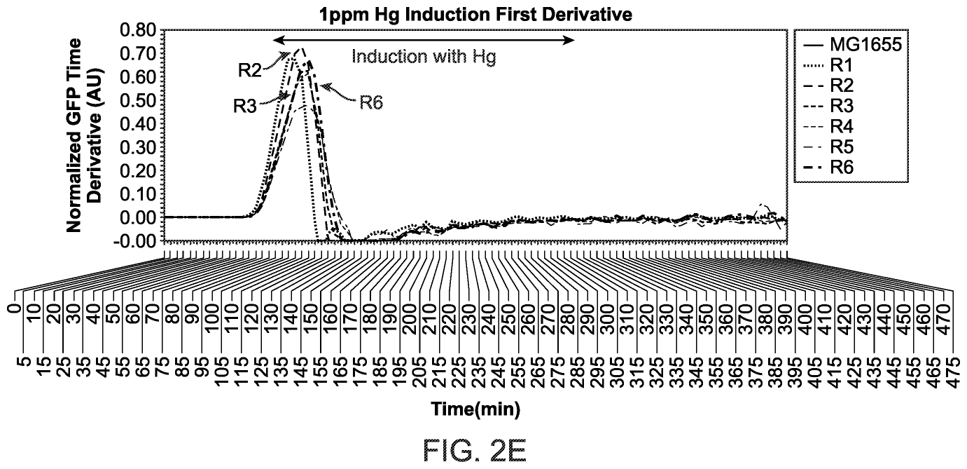

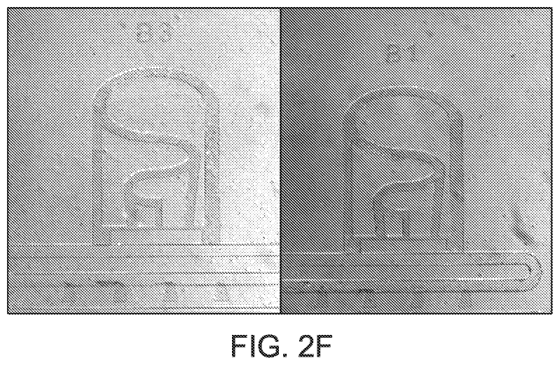

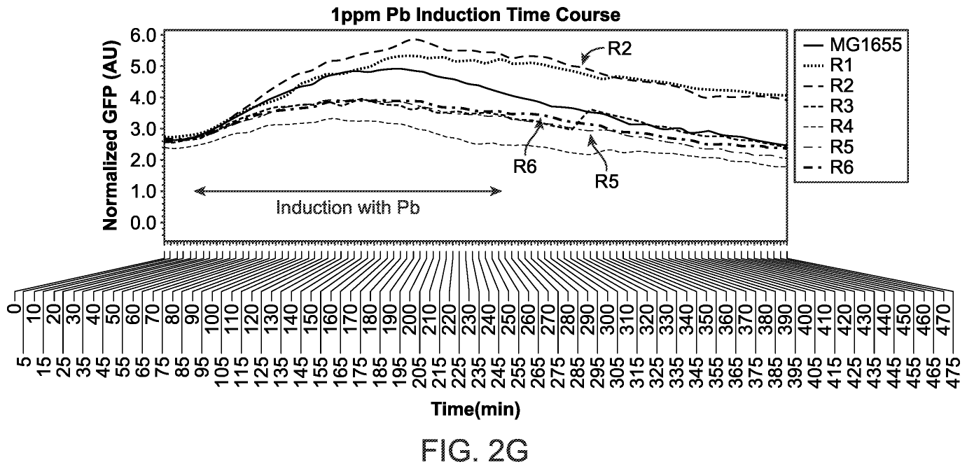

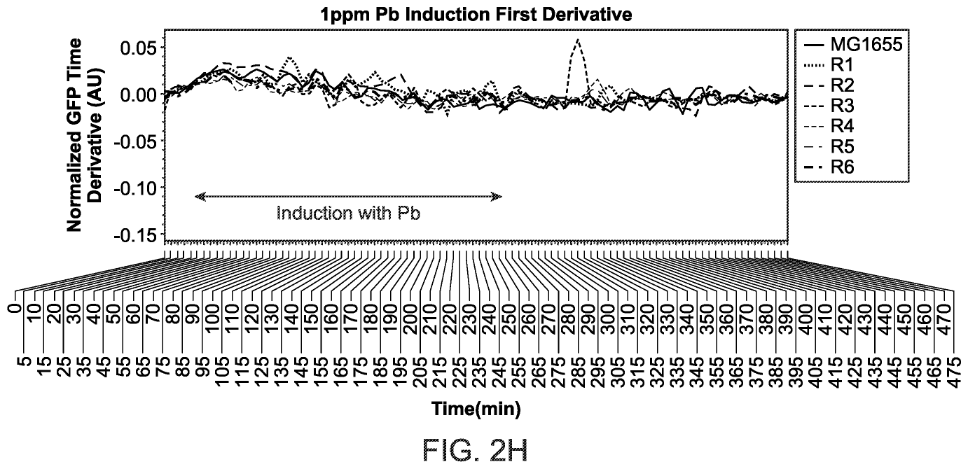

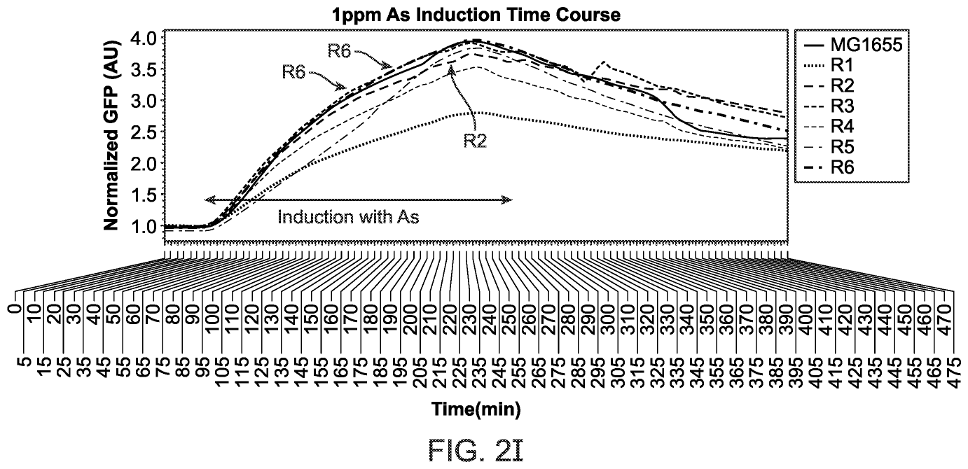

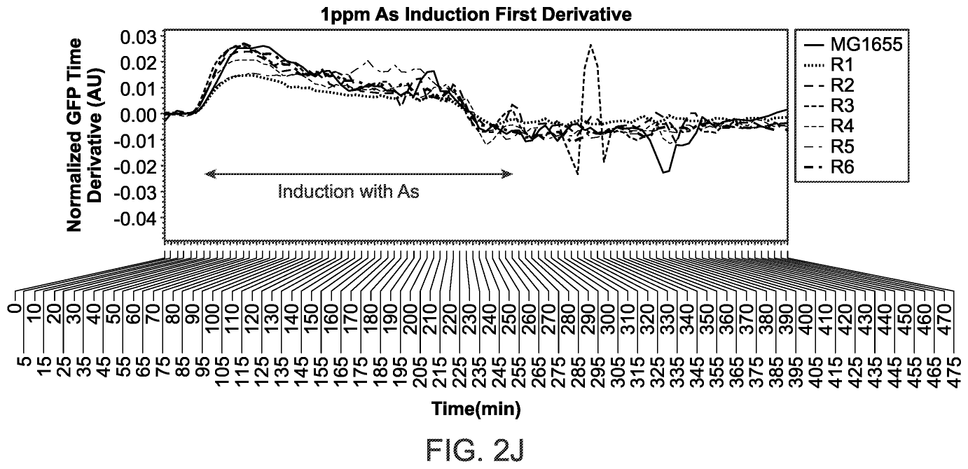

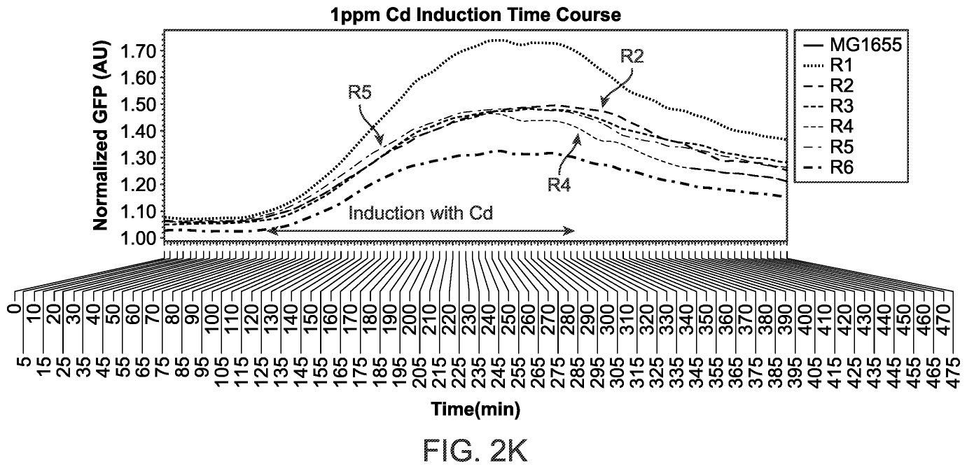

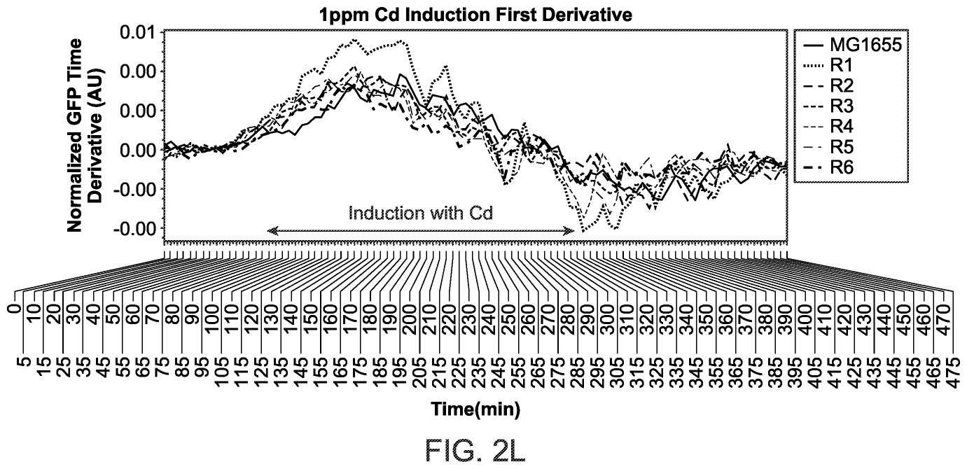

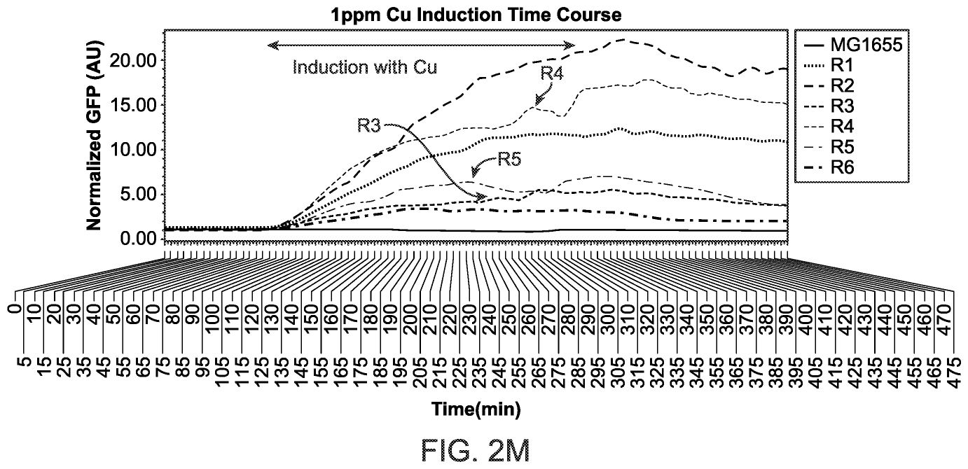

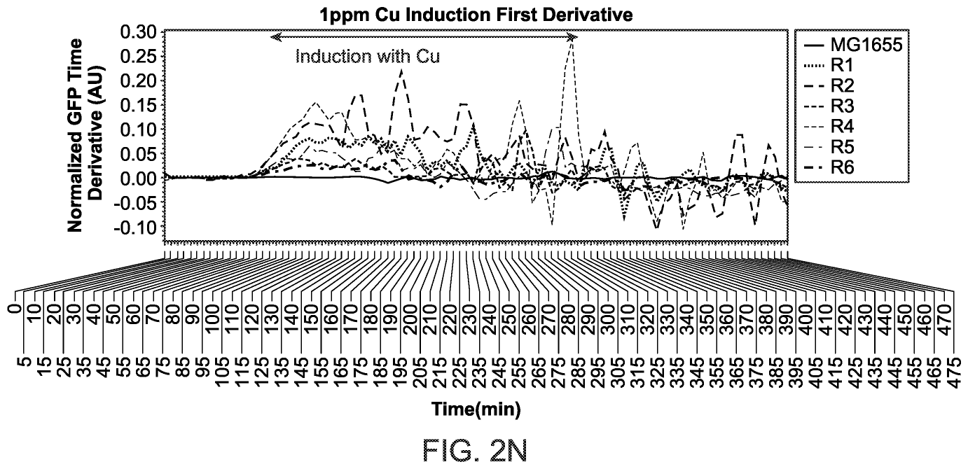

Microfluidics were used to compare the heavy metal response for a set of six adapted E. coli strain transformed with a heavy metal sensing plasmid. An exemplary microfluidic spotting region is shows in FIG. 2A and an exemplary microfluidic chip is shown in FIG. 2 Bwith an exemplary loading pattern for a series of adapted biosensing E. coli strains that sense a specific heavy metal shown in FIG. 2C.

FIGS. 2D and 2E show response curves of Hg3_MG1655 (non-adapted strain, also referred to as an ancestral strain) compared to Hg3_R1 through Hg3_R6. Responses were graph as background GFP expression subtracted and normalized by transmitted light values as a proxy for the amount of cells that have populated the microfluidic traps. In the responses to 1 ppm Hg there was a spectrum of fold-change response, relaxation, and response rate. The fold-change response refers to ratio of the maximal response upon induction to the basal GFP fluorescence level in the absence of inducer. The relaxation refers to the behavior of the sensor post-induction. The response rate refers to the slope of the response upon induction and is determined from the first derivative of the GFP fluorescence signal.

FIG. 2F shows that Hg3_MG presented a different GFP response profile and formed more biofilm in the channels with the HM9+50% seawater media when compared to Hg3_R2.

FIGS. 2G-2N show exemplary response curves for additional heavy metals with corresponding adapted biosensing E. coli strains.

For each of these response curves, the induction by the corresponding heavy metal yielded varied response phenotypes for each adapted biosensing E. coli strain. The variation in fold-change, response rate, and relaxation indicates that some of the adapted sensor strains have more desirable response behaviors, and in some cases, the evolved strains do not necessarily all exhibit better performance than the unevolved strain. However, this variability allows for selection of exemplary strains.

### Example 3—Identifying Exemplary Adapted Biosensing E. coli Strains

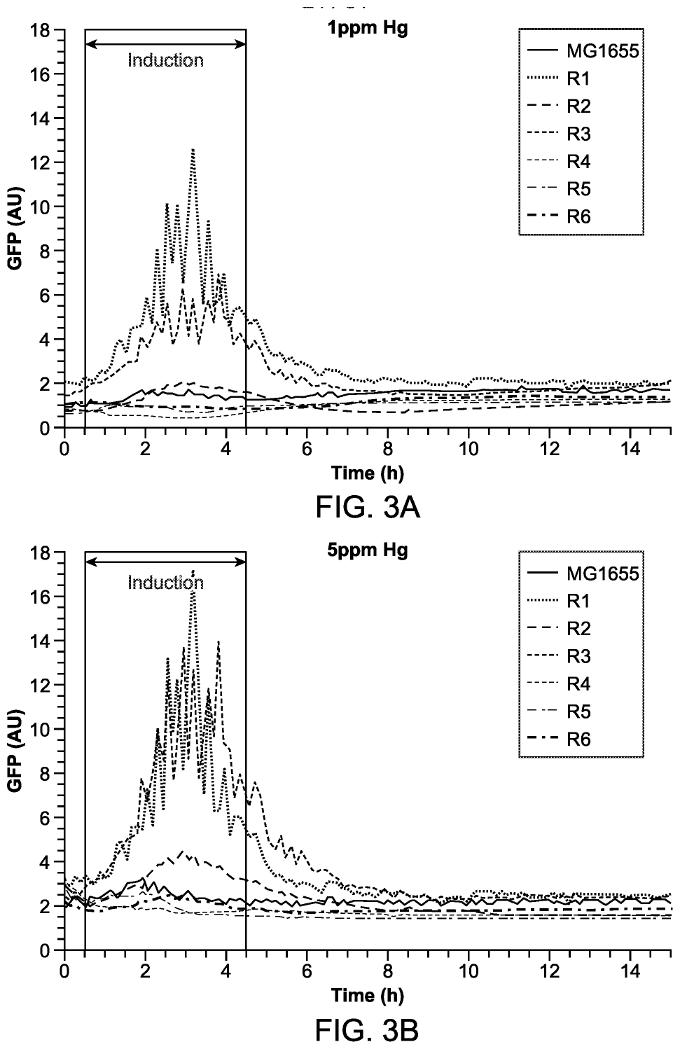

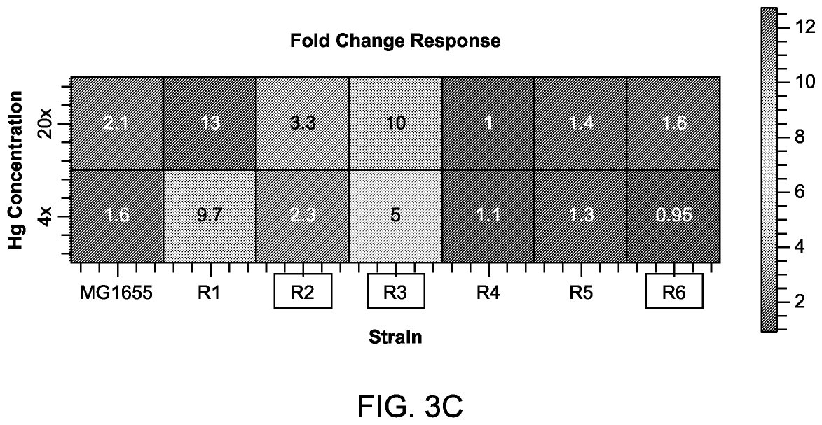

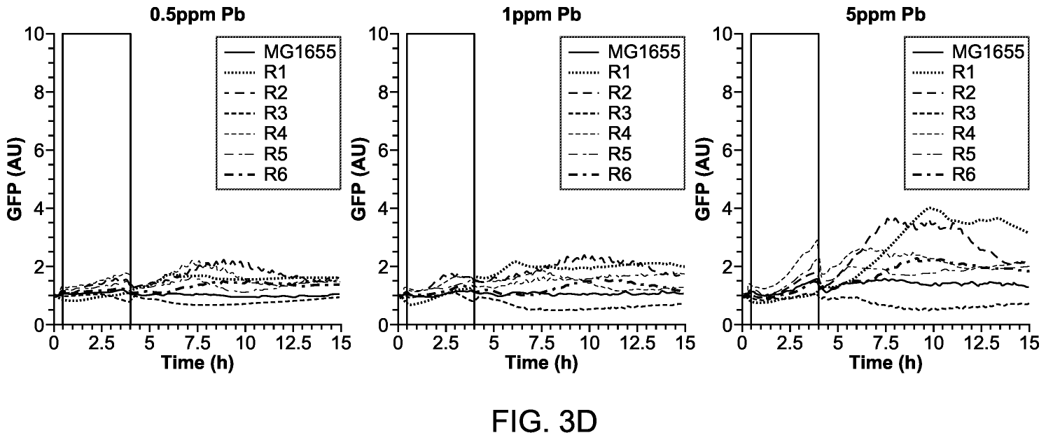

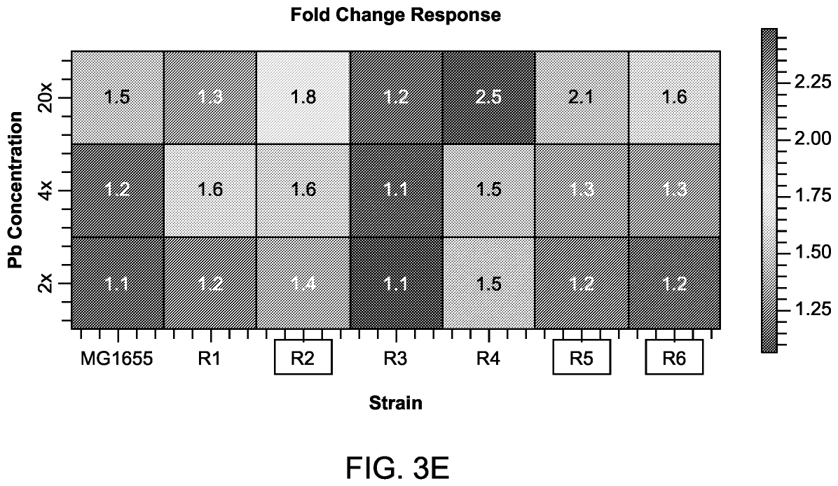

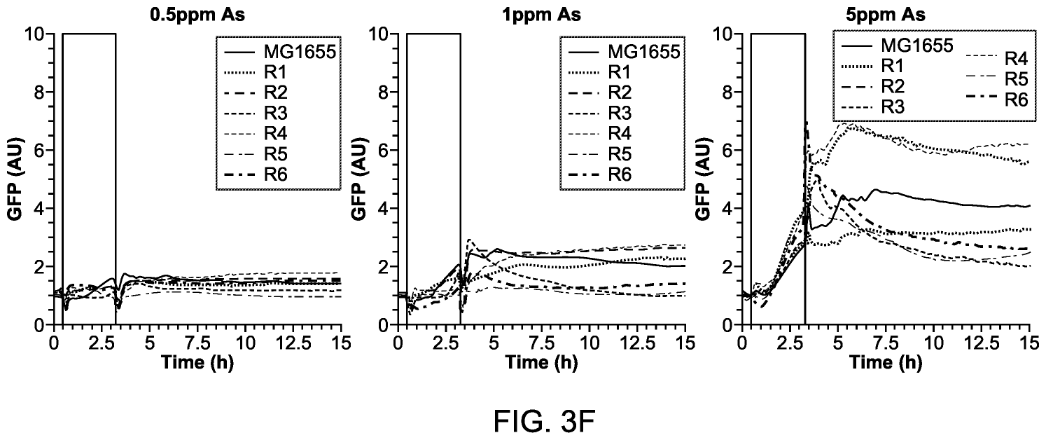

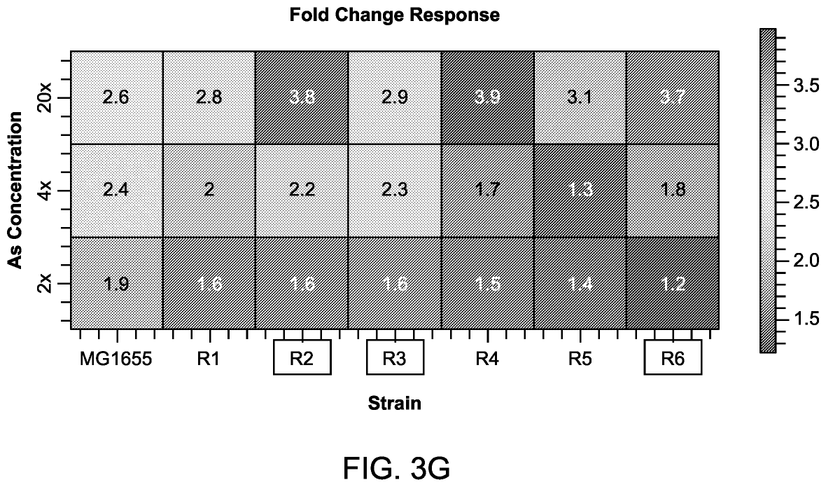

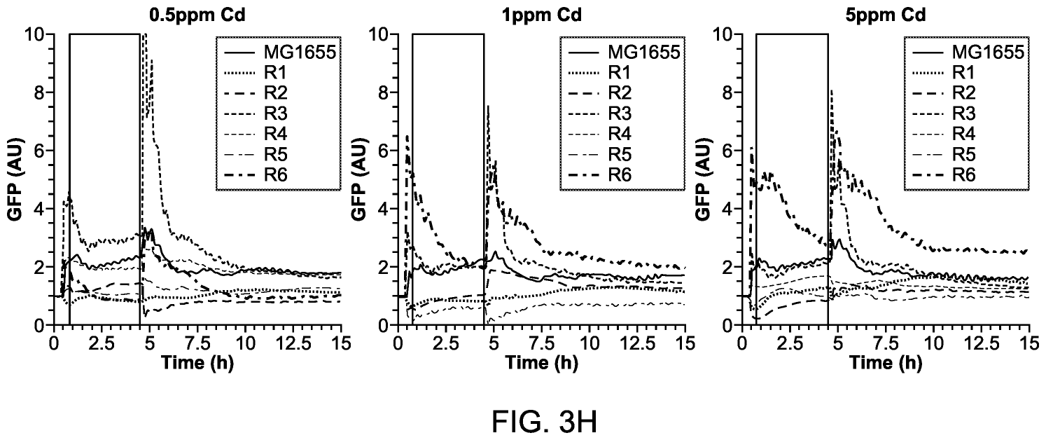

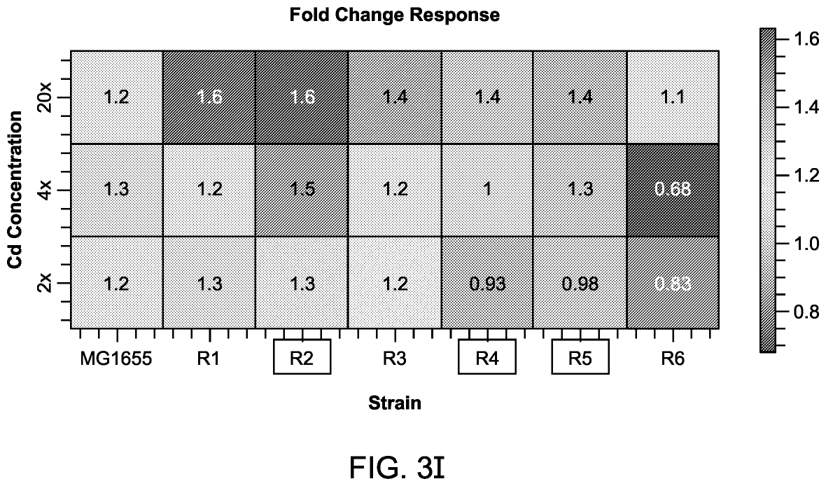

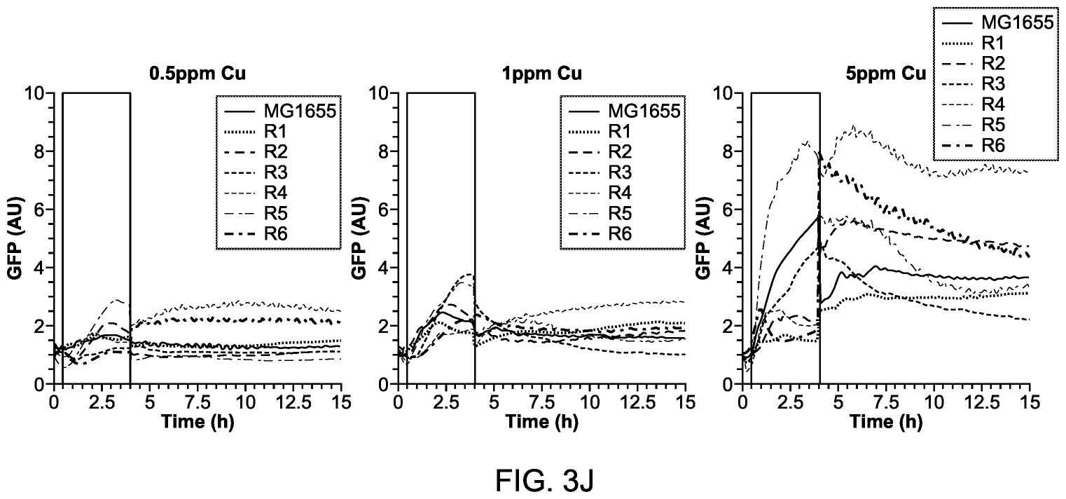

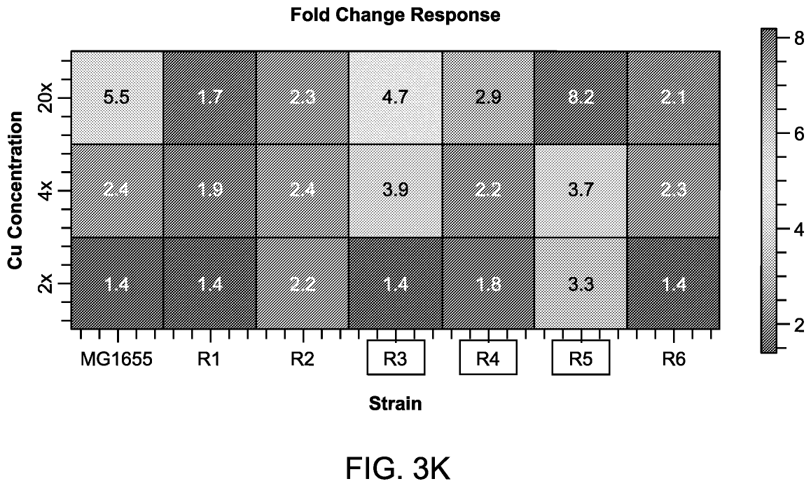

Adapted biosensing E. coli strains with more robust and sensitive responses were identified for each of arsenic, cadmium, copper, lead, mercury, and zinc, using Tecan plate readers to track GFP fluorescence response patterns when exposed to varying concentration of the heavy metal of interest. For each strain, optical density (OD) and GFP fluorescence were measured over several induction concentrations simultaneously (FIGS. 3A, 3B, 3D, 3F, 3H, 3J). By comparing fold change in responses (FIGS. 3C, 3E, 3G, 3I, 3K), exemplary strains were chosen for responding to various heavy metals in a heavy metal dose-dependent manner, with GFP fold-change corresponding to an increase in heavy metal concentration. Selected adapted biosensing E. coli strains were more responsive to a range of heavy metal concentrations and were heavy metal dependent (Table 6).

TABLE 6
Selected adapted biosensing E. coli strains.

| Heavy Metal | Selected Strain 1 | Selected Strain 2 | Selected Strain 3 |
|---|---|---|---|
| Arsenic | As7_R2 | As7_R3 | As7_R6 |
| Cadmium | Cd1_R2 | Cd1_R4 | Cd1_R5 |
| Copper | Cu3_R3 | Cu3_R4 | Cu3_R5 |
| Lead | Pb7_R2 | Pb7_R5 | Pb7_R6 |
| Mercury | Hg3_R2 | Hg3_R3 | Hg3_R6 |

### Example 4a—Using Adapted Biosensing E. coli Strain Heavy Metal Response Profiles and Machine Learning—Collecting Training Data

This example describes training data that was collected for a supervised machine learning classifier to identify the identity and concentration of heavy metals, such as arsenic, cadmium, copper, lead, mercury, and zinc, in an unknown sample.

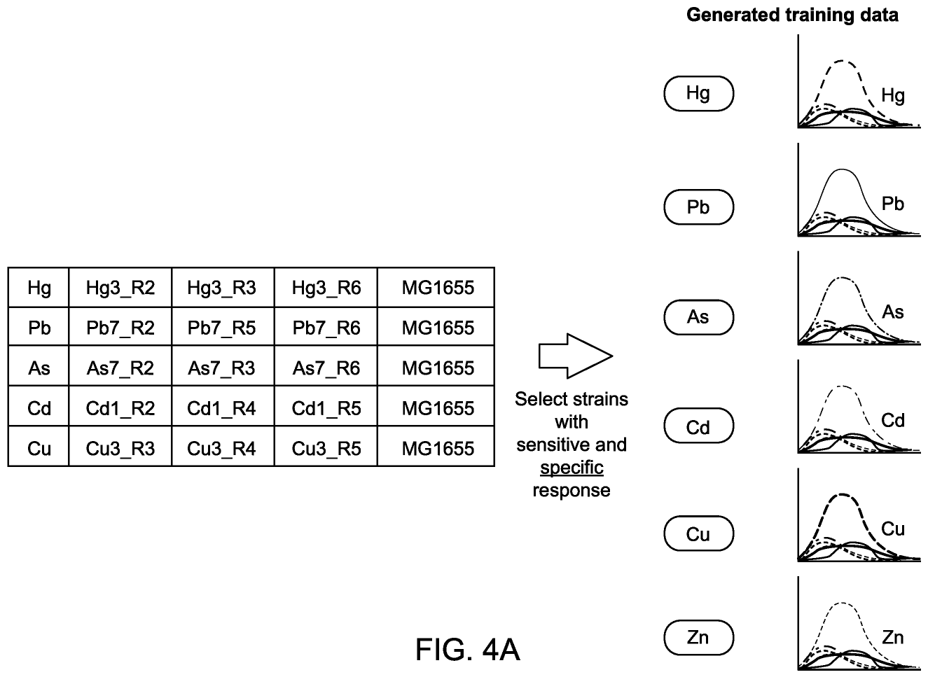

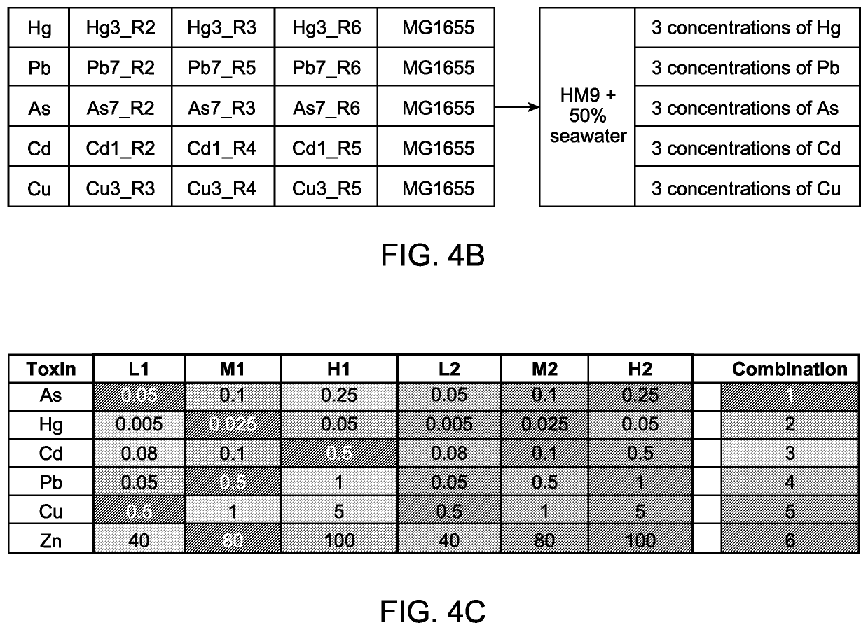

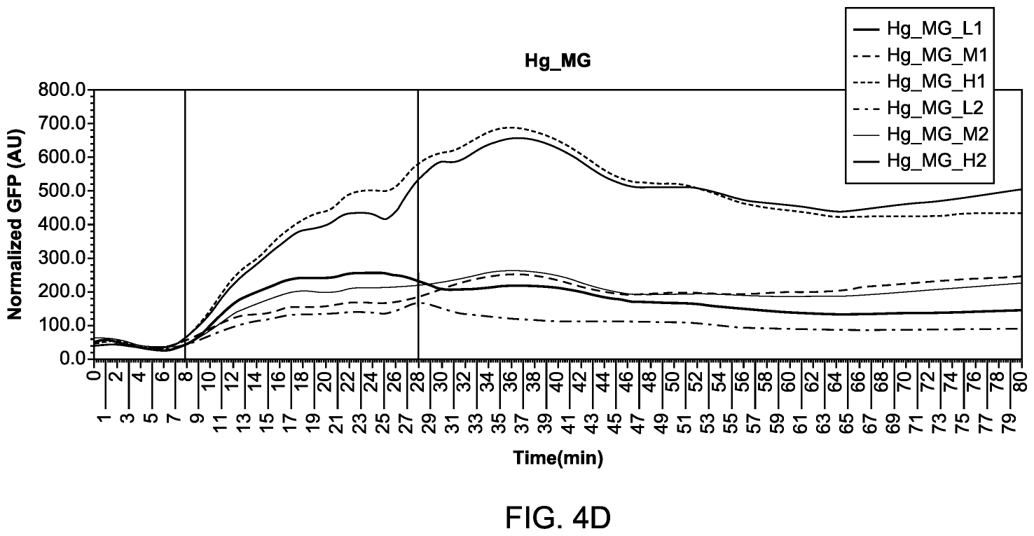

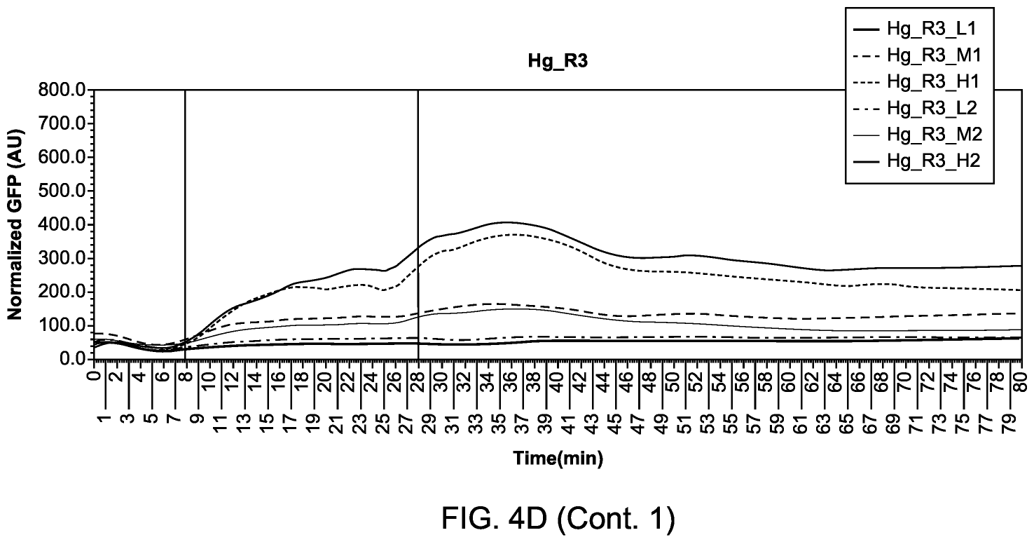

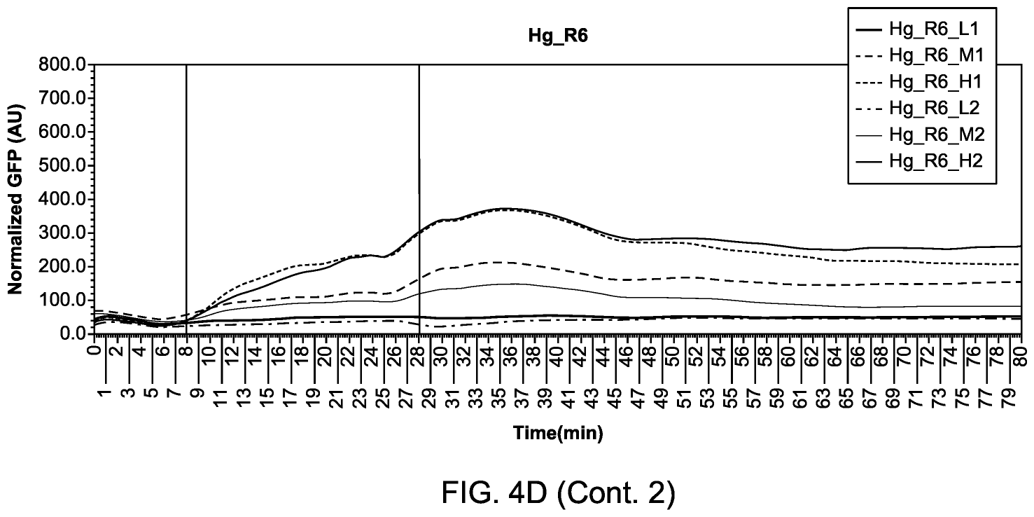

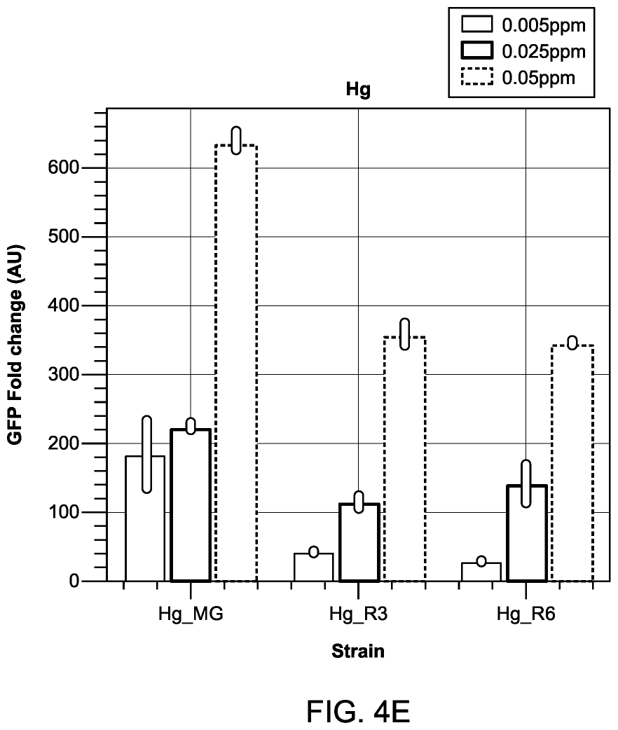

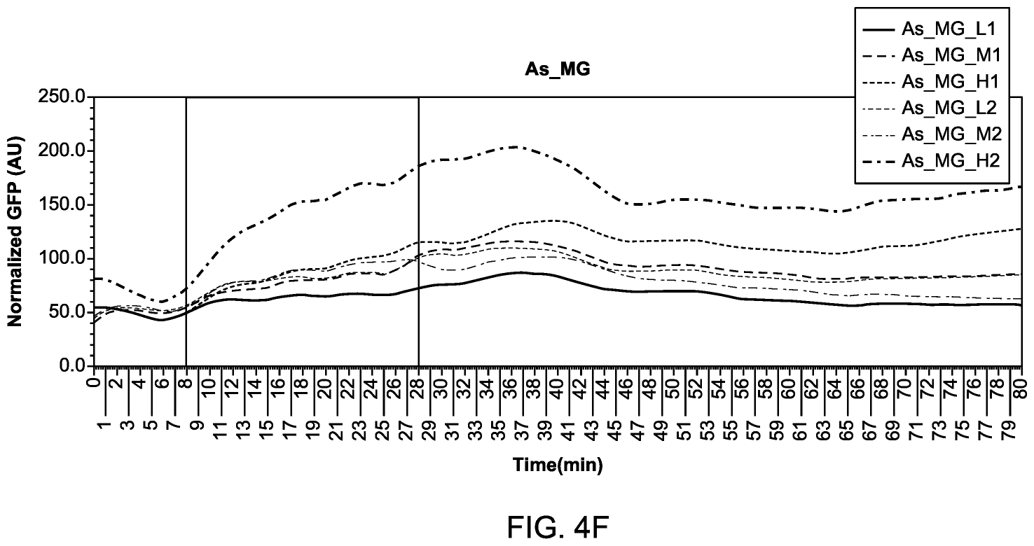

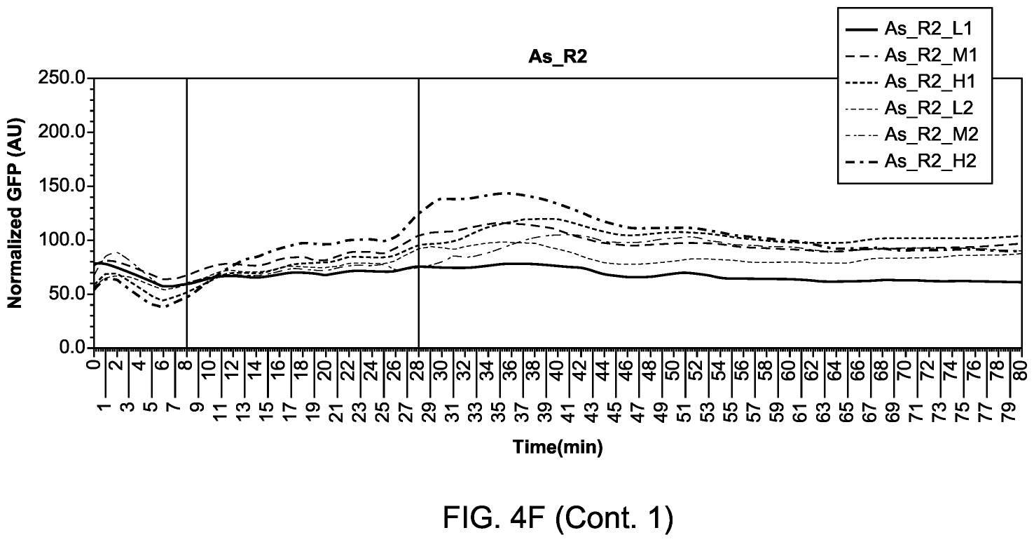

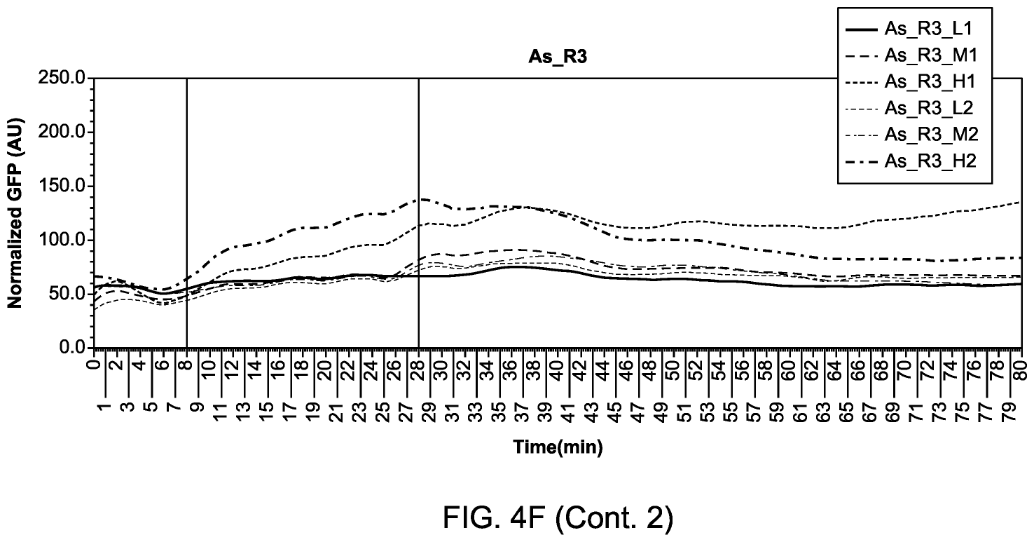

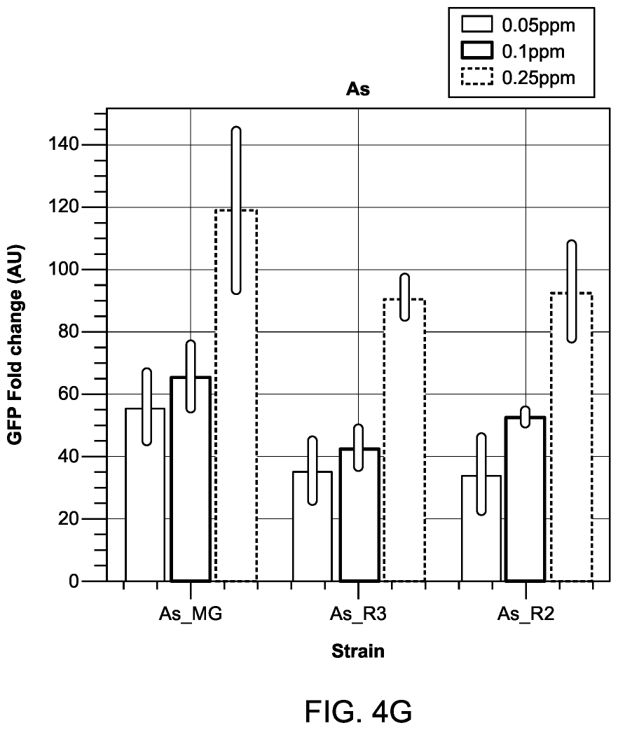

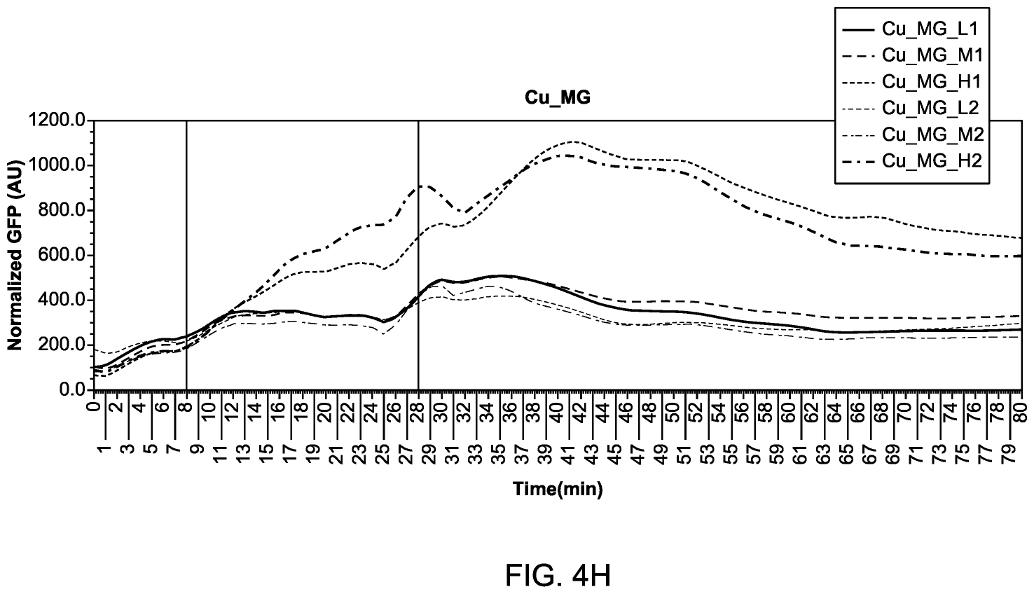

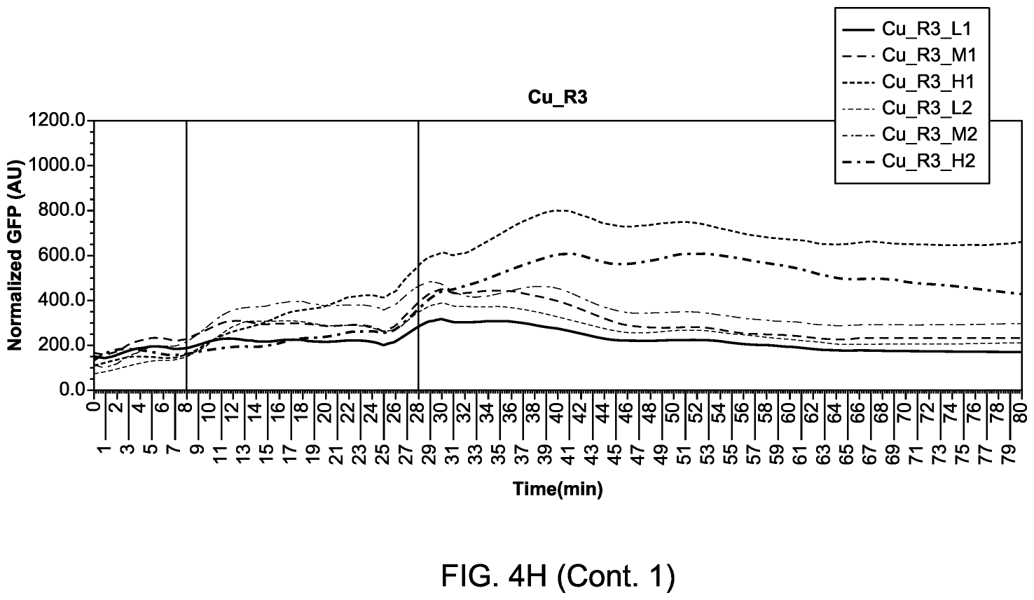

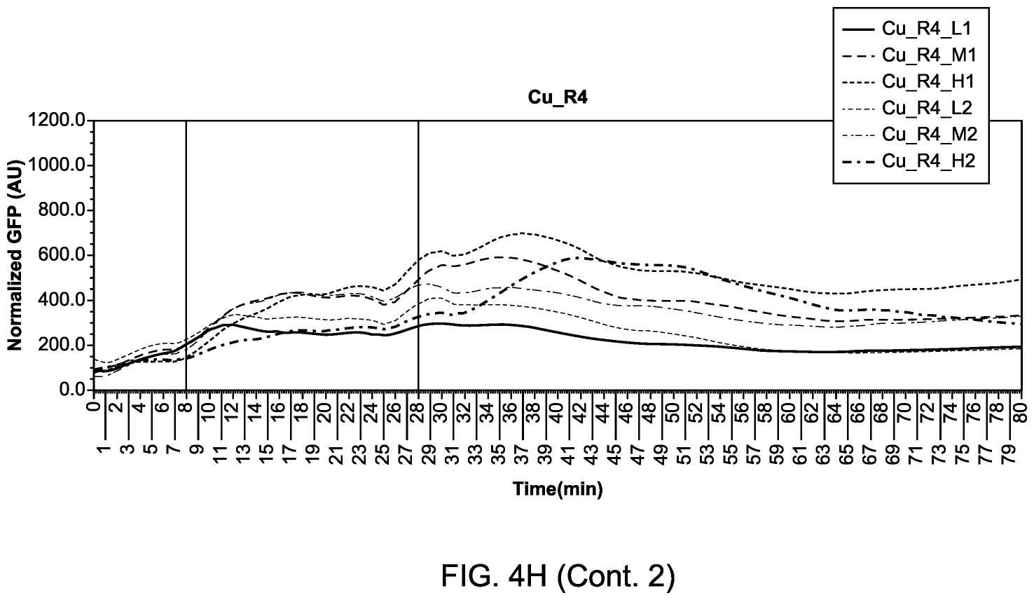

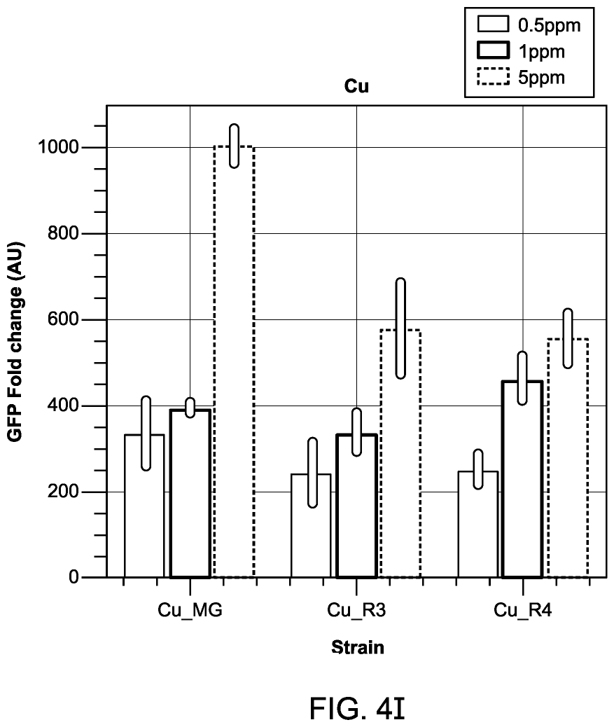

FIG. 4A depicts an exemplary workflow to generate training data from selected strains with sensitive and specific response patterns. A panel of selected adapted biosensing E. coli strains was grown in HM9+50% seawater, with each strain undergoing GFP induction for 80 minutes in at least three different concentrations of arsenic (0.0 5 ppm, 0.1 ppm, and 0.25 ppm), cadmium (0.08 ppm, 0.1 ppm, and 0.5 ppm), copper (0.5 ppm, 1 ppm, and 5 ppm), lead (0.5 ppm, 0.5 ppm, and 1 ppm), and mercury (0.005 ppm, 0.025 ppm, and 0.05 ppm) (FIGS. 4B-4C). All strains in FIG. 4B were subjected to all 6 combinations of heavy-metal induction indicated in FIG. 4C. Heavy metal combination contained arsenic, cadmium, copper, lead, mercury, and zinc in various combination to induce a variety of GFP responses.

Combination 1-0.05 ppm As, 0.025 ppm Hg, 0.5 ppm Cd, 0.5 ppm Pb, 0.5 ppm Cu, and 80 ppm Zn

Combination 2-0.1 ppm As, 0.05 ppm Hg, 0.1 ppm Cd, 0.5 ppm Pb, 1 ppm Cu, and 100 ppm Zn

Combination 3-0.25 ppm As, 0.005 ppm Hg, 0.08 ppm Cd, 1 ppm Pb, 5 ppm Cu, and 40 ppm Zn

Combination 4-0.05 ppm As, 0.05 ppm Hg, 0.08 ppm Cd, 0.5 ppm Pb, 1 ppm Cu, and 40 ppm Zn

Combination 5-0.1 ppm As, 0.005 ppm Hg, 0.08 ppm Cd, 0.05 ppm Pb, 5 ppm Cu, and 80 ppm Zn

Combination 6-0.25 ppm As, 0.025 ppm Hg, 0.1 ppm Cd, 1 ppm Pb, 0.5 ppm Cu, and 100 ppm Zn

Exemplary induction curves of Hg_R3 and Hg_R6 are shows in FIG. 4D, with a quantification of GFP fluorescence fold change showing that GFP fluorescence corresponded to increasing concentrations of Hg within the heavy metal combinations (FIG. 4E).

Exemplary induction curves of As_R2 and As_R3 are shows in FIG. 4F, with a quantification of GFP fluorescence fold change showing that GFP fluorescence corresponded to increasing concentrations of As within the heavy metal combinations (FIG. 4G).

Exemplary induction curves of Cu_R3 and Cu_R4 are shows in FIG. 4H, with a quantification of GFP fluorescence fold change showing that GFP fluorescence corresponded to increasing concentrations of Cu within the heavy metal combinations (FIG. 4I).

Hg, As, Cu adapted biosensing E. coli strains were sensitive and specific for sensing heavy metals combinatorially.

### Example 4b—Using Adapted Biosensing E. coli Strain Heavy Metal Response Profiles and Machine Learning—Determining Heavy Metal Identity and Concentration in Unknown Samples

An unknown sample of high salinity liquid is provided. The selected adapted biosensing E. coli strains are exposed to the unknown sample in an instrument that can determine new physiological measures, such as induction of GFP fluorescence fold-change, with excitation at 485 nm and emission at 510 nm (e.g., using a microfluidic device or a plate reader, equipped with a fluorescence sensor), response relaxation behavior, and response rate. In a microfluidic device, GFP induction can be measured by comparing an image taken before exposure to the unknown sample, and a second image taken after exposure to the unknown sample. The resulting GFP fold induction (input) and the training data from example 4a can be used by any appropriate machine learning algorithm to determine the identity of the heavy metal (output) and optionally the concentration of the heavy metal (output). Examples include supervised machine learning algorithms such as recurring neural networks (RNNs), long short term memory (LSTM), time series forest classifier (TSM), or shapelet-based classifier.

1. A method of determining an amount of an analyte in a sample using an adapted biosensing microbe, the method comprising:
a) inducing an adapted biosensing microbe in the sample, wherein the adapted biosensing microbe comprises an inducible promoter operably linked to a gene encoding a signal, wherein the inducible promoter is induced by the analyte to produce the signal;
b) measuring the signal; and
c) determining, in response to the signal, the amount of the analyte.

2. The method of claim 1, wherein the sample comprises a high salinity medium.

3. The method of claim 1, wherein the sample is of a sample type selected from groundwater, seawater, road run-off water, anaerobic bioreactor water, and sewer water.

4. The method of claim 1, wherein the analyte is a heavy metal selected from arsenic, cadmium, copper, lead, mercury, and zinc.

5. (canceled)

6. The method of claim 1, wherein the adapted biosensing microbe further comprises a modification in at least one of the genes selected from the group consisting of nagA, ompC, cueO, phoE, kup, treF, cca, and bacA, or homologous genes thereof, compared to the non-adapted microbe.

7. The method of claim 1, wherein the signal is a fluorescence signal.

8. The method of claim 1, wherein step (a) further comprises one or more of (i) inducing the adapted biosensing microbe in a microfluidic device; (ii) imaging the microfluidic device at least once while inducing or after inducing the biosensing microbe to generate at least one image; (iii) imaging the microfluidic device at least twice while inducing or after inducing the biosensing microbe to generate at least two images.

9. (canceled)

10. (canceled)

11. (canceled)

12. The method of claim 1, the signal can be used to determine a feature selected from the group consisting of a fold-change response, a final signal measurement, a maximum signal measurement, a signal relaxation response, and a signal response rate.

13. (canceled)

14. The method of claim 1, further comprises at least one of the group selected from:
i) generating a report comprising the determine amount of the analyte;
ii) storing data to a memory, the data comprising the determined amount of the analyte;
iii) transmitting an alert, the alert comprising the determined amount of the analyte; and
iv) initiating an automated device with automation instructions created based on the determined amount of the analyte.

15. The method of claim 1, further comprising determining the amount of the analyte comprises:
submitting to an amount classifier, as input, the signal, wherein the amount classifier is created via machine learning techniques selected from recurring neural networks, long short term memory, time series forest classifier, and shapelet-based classifier and trained on training data comprising training signals and training amounts, and
receiving, from the amount classifier as output, the amount of the analyte.

16. (canceled)

17. A nucleic acid construct comprising:
a) an inducible promoter selected from the arsR promoter, the cadC promoter, the cusC promoter, the zntA promoter, the mer promoter and the zraP promoter operably linked to a gene encoding a signal, wherein the inducible promoter is induced by a threshold level of a heavy metal selected from arsenic, cadmium, copper, lead, mercury, and zinc;
b) a selectable marker; and
c) an origin of replication.

18. (canceled)

19. (canceled)

20. The nucleic acid construct of claim 17, wherein the arsR promoter, the cusC promoter, and/or the zntA promoter is from an Escherichia coli (E. coli) strain.

21. (canceled)

22. The nucleic acid construct of claim 17, wherein the cadC promoter is from a Staphylococcus aureus (S. aureus) strain.

23. (canceled)

24. The nucleic acid construct of claim 17, wherein the mer promoter is bidirectional or from a transposon.

25. (canceled)

26. (canceled)

27. The nucleic acid construct of claim 17, wherein the signal is selected from a fluorescence signal, a luminescence signal, and a colorimetric signal.

28. (canceled)

29. (canceled)

30. The nucleic acid construct of claim 17, wherein the selectable marker confers resistance to ampicillin, kanamycin, chloramphenicol, erythromycin, spectinomycin, neomycin, streptomycin, zeocin, or gentamicin.

31. (canceled)

32. A biosensing microbe comprising the nucleic acid construct of claim 17.

33. (canceled)

34. The biosensing microbe of claim 32, wherein the microbe genera is selected from Saccharomyces, Pichia, Escherichia, Streptococcus, Staphylococcus, Salmonella, Campylobacter, Pseudomonas, Bacillus, Klebsiella, and Vibrio.

35. (canceled)

36. (canceled)

37. (canceled)

38. A method of producing an adapted biosensing microbe to detect an analyte in a sample, the method comprising:
a) obtaining a non-adapted biosensing microbe, wherein the non-adapted biosensing microbe comprises an inducible promoter operably linked to a gene encoding a signal, wherein the inducible promoter is induced by the analyte to produce the signal;
b) growing the non-adapted biosensing microbe in a medium similar to the sample to generate one or more adapted biosensing microbes; and
c) selecting the adapted biosensing microbe from the one or more adapted biosensing microbes by selecting for one or more traits selected from the group of increased growth rate compared to the non-adapted biosensing microbe, increased yield compared to the non-adapted biosensing microbe, increased sensitivity of detection of the analyte compared to the non-adapted biosensing microbe, increased production of the signal compared to the non-adapted biosensing microbe, decreased baseline of the signal in the absence of the analyte compared to the non-adapted biosensing microbe, decreased biofilm formation compared to the non-adapted biosensing microbe, increased synthesis of osmoprotectants compared to the non-adapted biosensing microbe, increased uptake of osmoprotectants compared to the non-adapted biosensing microbe, and increased resistance to toxicity associated with increased levels of osmoprotectants compared to the non-adapted biosensing microbe;
thereby producing an adapted biosensing microbe to detect an analyte in a sample.

39. The method of claim 38, wherein the analyte is a heavy metal selected from arsenic, cadmium, copper, lead, mercury, and zinc.

40. (canceled)

41. The method of claim 38, wherein the sample comprises high-salinity medium.

42. The method of claim 38, wherein the non-adapted biosensing microbe and the adapted biosensing microbe are of a genera selected from Escherichia, Streptococcus, Staphylococcus, Salmonella, Campylobacter, Pseudomonas, Bacillus, Klebsiella, Vibrio, Saccharomyces, and Pichia.

43. (canceled)

44. The method of claim 38, wherein after step (b), the method further comprises serially-passaging the adapted biosensing microbe at least once to fresh medium to generate a further adapted biosensing microbe.

45.-51. (canceled)
